(19) United States
(12) Patent Application Publication
(10) Pub. No.: US 2011/0117559 A1
(43) Pub. Date: May 19, 2011

(54) SMALL RNA DETECTION ASSAYS

(75) Inventors: Mark A. Behlke, Coralville, IA (US); Scott D. Rose, Coralville, IA (US); Kyle A. McQuisten, Iowa City, IA (US); Jeffrey A. Manthey, North Liberty, IA (US)

(73) Assignee: INTEGRATED DNA TECHNOLOGIES, INC., Skokie, IL (US)

(21) Appl. No.: 12/943,159

(22) Filed: Nov. 10, 2010

Related U.S. Application Data
(60) Provisional application No. 61/260,943, filed on Nov. 13, 2009.

(51) Int. Cl. C12Q 1/68

(52) U.S. Cl. 435/6

(57) ABSTRACT

The present invention comprises use of cleavable primers to perform qPCR detection of cDNA made from small RNA species. The cleavable primers offer improved specificity over standard PCR primers and are the method is compatible with a variety of methods to introduce priming sites at the 5′-end and 3′-end of the small RNA species.

### FIELD OF THE INVENTION

The present invention comprises use of cleavable primers to perform qPCR detection of cDNA made from small RNA species. The cleavable primers offer improved specificity over standard PCR primers and the method is compatible with a variety of approaches to introduce priming sites at the 5′-end and 3′-end of the small RNA species.

### BACKGROUND OF THE INVENTION

Small RNAs, such as microRNAs (miRNAs) or small interfering RNAs (siRNAs), regulate gene expression by targeting messenger RNAs for cleavage or translational repression, or altering transcription by silencing genes, or affecting chromatin structure (Ghildiyal, M. et al., Nat Rev Genet, 10, 94-108 (2009)). Small RNAs, therefore, play critical roles in cell proliferation, cell differentiation, and cellular responses. Mis-expression or mis-regulation of miRNAs is thought to be involved in a variety of disease states, including cancer (Croce, C. M., Nat Rev Genet, 10, 704-714 (2009)). Small RNAs are generated by specific enzyme complexes from much larger RNA precursors, and a mature small RNA has several key characteristic features such as a small size (generally about 20-30 nucleotides). Typically, miRNAs have a 5′ terminal monophosphate and a 3′ terminal hydroxyl group. Other classes of small RNAs can have different end structures; for example, Piwi small RNAs (piRNAs) have a 5′-hydroxyl and are 3′-end modified with a 2′-O-methyl RNA base. Other configurations may exist. Attempts to detect, quantify, and analyze mature small RNAs have been hindered by their small sizes, similarity between related yet distinct species, and, sometimes, attendant low copy numbers.

Northern blotting has been used to detect mature small RNAs (Sempere, L. F. et al., Dev Biol, 244, 170-179 (2002); Pall, G. S. et al., Nucleic Acids Res 35, e60 (2007)), but this method suffers from poor sensitivity and has very low throughput. Likewise, nucleic acid microarrays have been used to quantify mature small RNAs. This method has the advantage of very high throughput and the expression levels of a large number of different miRNA species can be studied simultaneously in a single sample (Nelson, P. T et al., Nat Methods, 1, 155-161 (2004); Liu, C. G. et al., Proc Natl Acad Sci U S A, 101, 9740-9744 (2004;)) Jacobsen et al., U.S. Patent Application 2005/0272075; Remacle et al., U.S. Patent Application 2006/0099619). However, this method suffers from difficulty with specificity and also requires a high concentration of input target for efficient hybridization; it is better suited to large surveys and not precise measurements of the expression levels of specific miRNAs of interest. Assays that directly identify unamplified miRNAs by a mass spectrometry signature have been devised (Griffey et al., U.S. Patent Application 2005/0142581), by direct visualization of adjacent two-color hybridization probes using single molecule detection (Neely et al., U.S. Patent Application 2006/0292616), or by direct single molecule sequencing (Kahvejian, UA20080081330); these methods require access to specialized equipment not available to most users.

Oligonucleotide ligation assays (OLAs) have been described where the small RNA (target) serves as a splint to position two synthetic probe oligonucleotides in a configuration suitable for ligation. The ligation event creates a larger molecule which can more easily be detected by various means, including radioactive detection (Maroney, P. A. et al., RNA, 13, 930-936 (2007))), bead capture with fluorescent imaging (Chen, J. et al., Nucleic Acids Res, 36, e87 (2008); Golub et al., U.S. Patent Application 2007/0065844; Han, U.S. Patent Application 2008/0166707), or PCR (Duncan, D. D. et al., Anal Biochem, 359, 268-270 (2006); Brandis et al., U.S. Patent Application 2006/0003337; Sorge et al., U.S. Patent Application 2006/0211000). The OLA class of assays are sensitive to mismatch at the site of ligation but are relatively insensitive to mismatches at other sites within the target, making the specificity of this assay good for some miRNAs but poor for others.

Direct PCR amplification of the small RNA would be a simple and accurate method to determine the presence and assess relative expression levels, however the short size of mature small RNAs precludes direct amplification by quantitative reverse transcriptase PCR (although the larger precursors may be PCR amplified). Methods have been developed to facilitate PCR detection of mature small RNAs. All of these methods require that additional sequence information is added to the small RNAs of interest, either by ligation or enzymatic extension, to provide for longer amounts of sequence to position primers for both PCR and reverse transcription (RT) steps. Ligation of synthetic oligonucleotides at both the 5′-end and 3′-end of the miRNA introduces a universal primer binding site to perform reverse transcription and then subsequently allows for use of small-RNA species-specific amplification primers that overlap both the linkers and the miRNA to perform quantitative PCR (qPCR). A variety of different formats have been devised to perform reactions of this kind, most of which vary in the method of attaching the terminal linkers (primer binding sites).

One method to provide PCR priming sites is to employ miRNA specific RT primers that partially overlap the 3′-end of the miRNA. Assays of this type have been described using linear primers or hairpin primers (Raymond, C. K. et al., RNA, 11, 1737-1744 (2005); Sharbati-Tehrani, S. et al., BMC Mol Biol, 9, 34; Chen et al., U.S. Patent Application 2005/0266418; Finn et al., U.S. Patent Application 2006/0078924; Tan et al., U.S. Patent Application 2007/0111226; Finn et al., U.S. Patent Application 2009/0087858). Once the 3′-linker is annealed to the small RNA, reverse transcription is performed. At this point qPCR can be directly performed using primers specific to the small RNA on one end and the small RNA plus linker on the other end. Alternatively, additional sequence can be attached as a 5′-linker to the opposing end of the small RNA, and qPCR can be performed as before. The reactions can be detected using SYBR™ Green or other dyes which permit detection of amplification products or using a fluorescent quenched probe positioned between the primers (e.g., a hydrolysis probe or a molecular beacon). Another method to attach a priming site at the 3′-end of the small RNA is enzymatic extension. Possibilities include use of terminal transferase or, preferably, poly-A polymerase (PAP) (Fu, H. J. et al., Mol Biotechnol, 32, 197-204 (2006); Fan et al, U.S. Patent Application 2008/0241831). Once tailing has been performed, the reverse transcription and qPCR can be performed as outlined previously. Attachment of priming sites onto the small RNA species and relative specificity of the ensuing qPCR reaction are potential weaknesses for the above methods. Methods involving species specific priming are difficult to multiplex and require a unique RT primer for each miRNA studied. Enzymatic elongation steps incorporate a homopolymeric sequence which has low complexity and complicates specificity of subsequent reactions.

The present invention comprises use of cleavable primers to perform qPCR detection of cDNA made from small RNA species. The cleavable primers offer improved specificity over standard PCR primers and the method is compatible with a variety of approaches to introduce priming sites at the 5′-end and 3′-end of the small RNA species.

### BRIEF SUMMARY OF THE INVENTION

MicroRNAs are short (typically 21-24 bases) and do not provide a sufficient length of nucleic acid to perform RT-qPCR reactions to assess identity or quantify the relative amounts of a given species present in a sample. Additional sequence information (such as oligonucleotide linkers) need to be added to at least the 3′-end or, preferably to both ends of the miRNA to introduce primer binding sites for both the RT and PCR phases of a detection reaction. Any RNA sample can be studied. Total RNA is preferred as procedures to subfractionate the RNA into subpopulations usually results in loss of material and adversely affects accurate quantification. It is preferable that the RNA be purified using Trizol, STAT-60, or other organic liquid phase based extraction procedure over a method using a rapid solid phase RNA binding column approach as the binding columns frequently result in significant loss of mass for the desired small RNA species.

In one embodiment of the invention, a 5′-adenylated synthetic oligonucleotide linker is attached to the 3′-end of the miRNA (or other small RNA species) using and RNA ligase such as T4 RNA Ligase in the absence of ATP. In addition to the 5′-adenylyl group, the linker oligonucleotide has a 3′-blocking group that prevents self ligation. Adenylation can be performed enzymatically or by chemical synthesis. The adenylated linker preferably comprises DNA bases; RNA bases can be employed but confer no added benefit. This method permits high efficiency linkering of RNA species without circularization, which is problematic with use of T4 RNA Ligase with ATP. Since miRNA species have a 5′-phoshate and 3′-hydroxyl, they readily undergo an undesired unimolecular circularization reaction under these conditions. The method employed herein avoids this problem by employing RNA Ligase without ATP, which permits ligation to the 3′-end of the receptor RNA species only when using an activated adenylated linker but does not permit ligation of nucleic acid species having only a 5′-phosphate. Following 3′-linkering, a second linker having a different sequence is attached to the 5′-end of the small RNA. This linker has a 3′-hydroxyl and is blocked at the 5′-end to prevent self-ligation reactions. This linker is partially or entirely made of RNA bases. For efficient ligation employing T4 RNA Ligase, the 3′-receiving nucleic acid must be RNA and maximal efficiency is achieved if 10-15 bases of RNA are present. The linkering reaction as outlined above will support detection of any small RNA species present in a heterogeneous sample that has a 5′-phosphate and 3′-hydroxyl. Variations in this scheme will permit detection of RNA species having different composition (see U.S. Patent Application 2009/0011422).

Following 5′ and 3′ linkering, the target RNA is converted to cDNA by reverse transcription using a DNA primer complementary to the 3′-linker. This primer is “universal” in that it does not contain any sequence specific to an individual small RNA species and simply anneals to the 3′-linker. The cDNA product can now be used as target in a qPCR reaction.

Optionally, small amounts of a synthetic single-stranded RNA oligonucleotide can be added to the linkering reaction (“spiked” into the total RNA sample) which serves as a positive control for the linkering process. This synthetic RNA oligonucleotide will mimic natural miRNAs, having a 5′-phosphate and 3′-hydroxyl and is preferably 21-24 bases in length. The sequence is selected to have no significant homology to any known small RNA species and is detectably different from other known RNA species in subsequent qPCR reactions. Assays specific to the synthetic “spike” can be used (see below) to demonstrate that successful 3′ and 5′ linkering and cDNA conversion has been achieved and further can be used to quantitatively assess the relative efficiency of these steps.

A novel cleavable-primer PCR method is used to perform the qPCR phase of the small RNA detection method of the present invention. The method employs the polymerase chain reactions (PCR) and can be performed in both end-point and real-time modes; it is quantitative when performed with known mass standards and run in real-time mode. The method employs blocked primers or primers which are otherwise designed to be incompetent to prime DNA synthesis. A cleavable linkage is positioned at or near the 3′-end of the primer. In one embodiment, this cleavable linkage is an RNA base. Following hybridization to a complementary sequence, the terminal blocking group or structure that prevents priming is removed by action of a thermostable RNase H2 enzyme (the “unblocking enzyme”). The unblocking reaction requires that the primer be in duplex form and moreover is sensitive to correct base pairing such that unblocking is inhibited by the presence of a base mismatch in the vicinity of the cleavage site. Through this mechanism, the blocked primers provide improved specificity when compared with traditional priming methods, resulting in lower background, reduced mispriming, and eliminating primer-dimer formation. With lower background, a greater number of PCR cycles can be run, giving the potential for increased sensitivity. The assay can be run using SYBR™ Green detection or other similar dye binding methods. Alternatively, one of the cleavable primers can be modified to have at least one reporter dye and at least one dark quencher on opposite sides of the cleavage site. In this configuration, the labeled primer-probe is dark in single-stranded conformation. Hybridization and cleavage separates fluorophore from quencher, permitting detection of the fluorophore. The cleavable primer reaction scheme is shown in FIG. 1.

The cleavable primers are positioned such that the 5′-end of the primer lies within sequence encoded by the synthetic linkers. The precise length of primer that extends into this domain can be varied to adjust the precise melting temperature (Tm) of the primer so that all primers can have a similar Tm, typically around 60° C. under standard conditions (see below). The 3′-end of the primer lies within the small RNA encoded sequence and is positioned such that at least 1-2 bases of primer remains in the small RNA domain following cleavage at the ribonucleotide residue. The precise position of the 3′-end within the small RNA/miRNA sequence can be uniquely adjusted for each sequence to maximize specificity, positioning sites of maximum variation at the cleavable ribonucleotide and what becomes the 3′-terminal of the primer following cleavage. Once the 3′-end of the primer has been defined, position of the 5′-end of the primer is adjusted to extend as far as needed into the linker domain to balance Tm at or around 60° C. under standard conditions.

Thus a single universal primer is employed for the RT reaction and unique sets of Forward/Reverse (for/rev) cleavable primers are needed for each small RNA species detected. The overall scheme for linkering, reverse transcription, and qPCR employing cleavable primers is shown in FIG. 2.

A control qPCR assay is envisioned to detect the positive control “spike” RNA described above. This can be run in parallel with other small RNA-specific reactions as a positive control for the linkering reactions and to permit relative measurement of the efficiency of the sample preparation steps of the miRNA detection process.

### BRIEF DESCRIPTION OF THE DRAWINGS

FIG. 1 is a reaction schematic of RNase H2 activation of blocked PCR primers.

FIG. 2 is a reaction schematic showing the steps involved in qPCR detection of small RNA species in heterogeneous RNA samples including: 1) a 3′-Linkering reaction, 2) a 5′-Linkering reaction, 3) a cDNA synthesis reaction, and 4) qPCR detection using RNase H2 cleavable primers.

FIG. 3 is the reaction schematic of FIG. 2 replaced with the actual linker and probe sequences employed to detect miR-16 in Examples 1, 2, and 3. DNA bases are uppercase, RNA bases are lowercase, “p” is phosphate, “x” is a C3 propanediol aliphatic spacer. Linker derived sequences are underscored.

FIG. 4 shows two variants of RNase H2 cleavable primers that could be used in a miR-16 detection assay (top and middle) and the activated primer that results following RNase H2 cleavage (bottom). An arrow indicates the location of RNase H2 cleavage. DNA bases are uppercase, RNA bases are lowercase, “p” is phosphate, “x” is a C3 propanediol aliphatic spacer. Linker derived sequences are underscored.

FIG. 5 is an amplification plot for the miR-16 detection assay depicted in FIG. 3 performed using a synthetic cDNA template, which mimics the expected product of the linkering and cDNA synthesis steps when performed on natural miR-16 RNA and serves as a positive control for the miR-16 cleavable primer assay. This target (SEQ ID No. 7) can be employed to make a quantification standard curve. Cycle number is shown on the X-axis and relative fluorescence intensity is shown on the Y-axis. Reactions performed using unmodified control primers and blocked RNase H2 cleavable primes are shown, with and without RNase H2 in the reaction mix.

FIG. 6 shows amplification plots for the miR-16 detection assay depicted in FIG. 3 using a synthetic miR-16 RNA oligonucleotide. All assay steps including 1) the 3′-Linkering reaction, 2) the 5′-Linkering reaction, 3) the cDNA synthesis reaction, and 4) qPCR detection using RNase H2 cleavable primers were performed. The top panel shows negative control reactions (12 total) where the linkering and cDNA steps were performed on a mock sample with no input RNA. The bottom panel shows triplicate reactions performed on 10⁻³, 10⁻⁴, 10⁻⁵, and 10⁻⁶ dilutions of the cDNA product produced from linkering reactions done using the synthetic miR-16 RNA.

FIG. 7 shows an amplification plot for the miR-16 detection assay depicted in FIG. 3 performed using total HeLa cell RNA. PCR reactions were performed in triplicate and the curves showing positive miR-16 detection represent signal from 0.4 ng cDNA equivalent. The negative control reactions show no detectable signal using the miR-16 specific primers using samples where the linkering and cDNA synthesis steps were performed without addition of HeLa RNA.

### DETAILED DESCRIPTION OF THE INVENTION

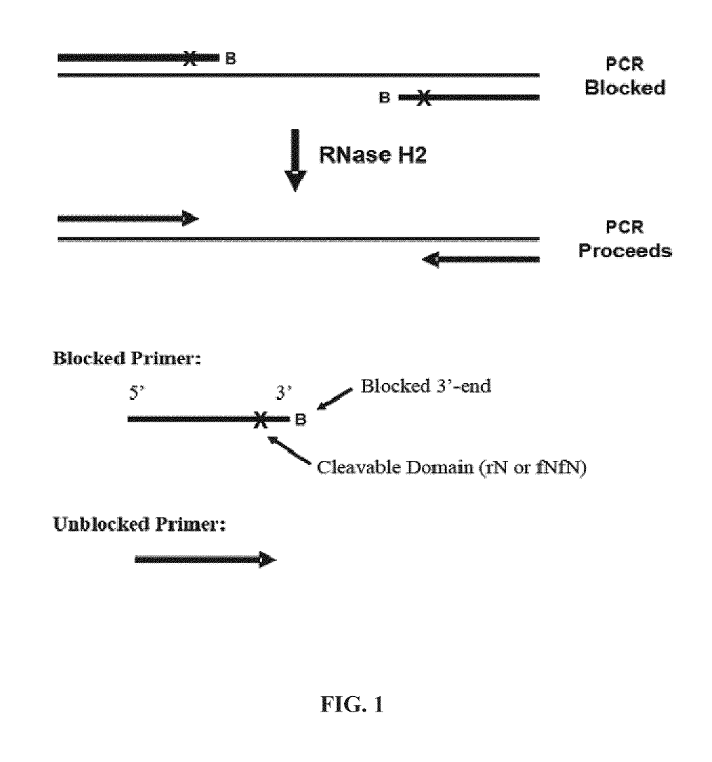

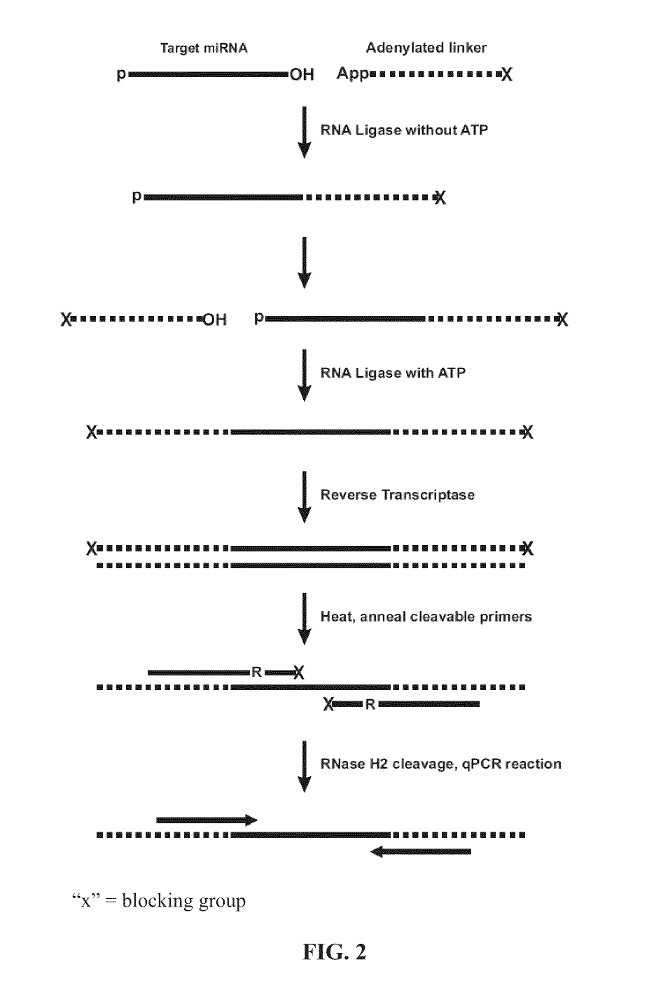

The invention provides for the use of blocked primers that are incompetent to support PCR until unblocked by action of a cleaving enzyme when hybridized to a perfect or near perfect match target. In one embodiment of the invention, the primers contain a single RNA residue at or near the 3′-end which comprise a cleavable site and the cleaving enzyme is RNase H2. For the detection of small RNA species, the target is a cDNA copy of the small RNA produced via a reverse transcription reaction preferably employing a synthetic universal primer site attached to the 3′-end of the small RNA by ligation or synthesis. In subsequent PCR steps, synthetic nucleic acid sequences are added to the small RNA species at the 5′-end by ligation so that primer biding sites of suitable length and base composition are present at both the 5′-end and the 3′-end of the small RNA sequence to support a PCR reaction. The method of using RNase H2 cleavable blocked primers in PCR is shown in FIG. 1. The flow for one embodiment of the detection method including linkering, reverse transcription, and qPCR steps is shown in FIG. 2.

A variety of methods can be used to attach said linker sequences. One method comprises a poly-A polymerization step using the enzyme Poly-A Polymerase (PAP) followed by a universal primer-mediated cDNA synthesis (reverse transcription) reaction. Although the PAP reaction is robust, this method only serves to add a homopolymeric poly-A tail onto the small RNA species which limits both the complexity and thermal stability (melting temperature, or Tm) of primers binding to that region.

A more preferred embodiment employs an RNA Ligase enzyme (such as T4 RNA Ligase) to covalently attach a linker of defined sequence to the 3′-end of the small RNA species. In one embodiment, this approach proceeds in two phases. In the first phase a first synthetic oligonucleotide linker is attached to the 3′-end of the small RNA. In the second phase, a second synthetic oligonucleotide linker is attached to the 5′-end of the small RNA. Direct use of T4 RNA Ligase to attach a linker to the 3′-end of the small RNA is problematic if the target is a miRNA species as this class of small RNA has a 5′-phosphate and 3′-hydroxyl. This configuration allows for efficient circularization of the target RNA in a unimolecular reaction in favor of the desired bimolecular reaction between target RNA and linker. The present embodiment of the invention preferably employs adenylated linkers and further employs T4 RNA Ligase without ATP in the reaction mixture. The use of T4 RNA Ligase plus ATP is typically employed to perform single-stranded RNA ligation reactions; however this mixture will circularize the small RNA. In the biochemical reaction, T4 RNA Ligase uses ATP to adenylate the 5′-end of the “donor” species of the ligation reaction. The adenylated species is an activated molecule and is competent to proceed with ligation in the absence of any additional ATP. If an adenylated linker is employed in the reaction mixture, then the ligation reaction can proceed in the absence of ATP. In the absence of ATP, T4 RNA Ligase will not circularize the small RNA; however, the desired ligation event can still proceed, resulting in the 5′-end of the linker being covalently attached to the 3′-end of the miRNA. Use of adenylated linkers for high efficiency coupling to miRNA without loss of target miRNA to circularization was taught by Lau (Science, 294, 858-862 (2001)) and is part of the method of the miRCat miRNA Cloning Kit (Integrated DNA Technologies, Coralville, Iowa, USA) as described in Devor (Devor et al., U.S. Application 2009/0011422).

Typically the activated cloning linker used in the 3′-linkering reaction comprises a 5′-adenylyl group, a suitable number of internal nucleic acid bases to function as a site for hybridization of RT and/or PCR primers, and a 3′-blocking group. The 3′-blocking group prevents the linker from itself participating in a circularization reaction or forming concatamers by serial head-to-tail additions. Suitable 3′-blocking groups include phosphate, aliphatic spacers (e.g., propanediol), a 3′-terminal dideoxy base, or other such strategies as are well known to those skilled in the art. The linker will have sufficient length and sequence complexity to provide for a suitable priming site for both RT and PCR reactions and will typically be between 15 and 25 bases long. The linker can comprise DNA, RNA or modified or non-natural bases. DNA bases are more stable and less expensive to manufacture and are preferred for the adenylated 3′-linker. Ligation efficiency is similar whether the linker is DNA or RNA. Linkers employed for cloning small RNA species can contain restriction enzyme cleavage sites to facilitate subsequent ligation and cloning reactions. For detection assays as taught herein, restrictions sites are not necessary and can be deleterious as such sites are typically palindromic in nature and contribute to hairpin and self dimer binding potential, which is undesirable for primer sequences. Ideally the synthetic oligonucleotide employed as a 3′-linker will be pre-screened to have minimal homology to known RNA species within the target genome of interest and offers a unique priming site. Small RNA species of interest are cataloged in miRBase (http://www.mirbase.org/) and other sites known to those with skill in the art.

Small RNA species in natural samples exist in heterogeneous mixtures comprising a variety of different small RNAs, longer messenger RNA (mRNA), transfer RNA (tRNA), and ribosomal RNA (rRNA) species. The small RNAs comprise a small fraction of the RNA mass present. The RNA sample is purified from the original biological source (e.g., cells, tissue, etc.) using methods where care is taken to maintain the presence of small species. Liquid organic extraction methods are generally preferable to solid phase binding resins for isolation of RNA as binding resin methods typically enrich for long RNAs and small RNA species are frequently depleted or entirely lost.

A 3′-end linkering reaction is performed on the RNA sample. The adenylated linker is introduced in molar excess into a reaction mixture with the biological RNA sample in a buffer suitable for activity of the precise enzyme employed. An RNA Ligase is added to the mixture, such as T4 RNA Ligase, in the absence of ATP and the reaction is incubated for a suitable length of time to ensure the ligation reaction is complete or nearly complete. At this point the reaction products can be purified by electrophoresis, HPLC, or other means. However purification results in loss of sample and is not necessary; it is preferable to directly proceed to the next steps in the detection process.

At this point it is possible to perform reverse transcription to convert the linkered small RNA species to cDNA. Alternatively, a 5′-linker can be attached to the 5′-end of the small RNA-3′-linker species. In one embodiment of the invention, a 5′-linkering reaction is performed immediately following the 3′-linkering reaction without the need to exchange buffers. In this process, the 5′-end of the small RNA is ligated to a second synthetic oligonucleotide linker, using an RNA Ligase enzyme.

The 5′-linker has a 3′-hydroxyl which is necessary for the ligation reaction. The 5′-linker may have a 5′-hydroxyl or preferably will have a 5′-modifying group that blocks the 5′-end of this species from participation in ligation reactions. Suitable blocking groups include aliphatic spacers (e.g., propanediol), amino-modifiers, or any of a variety of groups well know to those with skill in the art. A 5′ phosphate group is undesirable as this will promote ligation reactions involving the 5′-end of the 5′-linker. The 5′-linker can have sufficient length and sequence complexity to provide a suitable priming site for PCR reactions and will typically be between 15 and 25 bases long. The 5′-linker is comprised of RNA, DNA or non-natural bases and can be optimized as needed with the RNA ligase used. In one embodiment, the 5′-linker is preferably RNA or is chimeric comprising both DNA and RNA bases with RNA bases positioned towards the 3′-end. For reactions employing T4 RNA Ligase, ligation efficiency is improved if the 5′-nucleic acid species comprises RNA bases for 10-15 residues at its 3′-end. Linkers employed for cloning small RNA species will contain restriction enzyme cleavage sites to facilitate subsequent ligation and cloning reactions. For detection assays as taught herein, restrictions sites are not necessary and can be deleterious as such sites are typically palindromic in nature and contribute to hairpin and self dimer binding potential, which is undesirable for primer sequences. Ideally the synthetic oligonucleotide employed as a 5′-linker will be pre-screened to have minimal homology to known RNA species in the target organism of interest and offers a unique priming site. Small RNA species of interest are cataloged in miRBase (http://www.mirbase.org/) and other sites known to those with skill in the art.

The 5′-linker is introduced in molar excess into a reaction mixture with the biological RNA sample in a buffer suitable for activity of the precise enzyme employed. An RNA Ligase is added to the mixture, such as T4 RNA Ligase, in the presence of ATP and the reaction is incubated for a suitable length of time to ensure the ligation reaction is complete or nearly complete. If the 5′-linkering reaction is conducted in the same mixture as the 3′-linkering reaction, then T4 RNA Ligase is already present and only the 5′-linker oligonucleotide and ATP must be added to complete the new reaction mixture. Following incubation, the reaction products can be purified by electrophoresis, HPLC, or other means. However purification results in loss of sample and is not necessary; it is preferable to directly proceed to the next steps in the detection process.

At this step of the preferred method, the small RNA species is flanked on the 5′-end by the 5′-linker, which itself is RNA or mostly RNA, and on the 3′-end by the 3′-linker, which is RNA or DNA. This species is converted to cDNA by reverse transcription using the 3′-linker as a universal primer binding site. A synthetic DNA oligonucleotide that is complementary to the 3′-linker serves as the RT primer. This primer is preferably not specific for any particular small RNA species and is universal in that it will serve to prime a cDNA synthesis reaction for any RNA species covalently attached to the 3′-linker. Reverse transcription is performed using routine methods known to those skilled in the art.

The identity and relative amount of the miRNA species is next determined by a qPCR reaction using the cDNA produced above as template. QPCR reactions specific for the miRNA require use of two miRNA species-specific primers. A unique primer pair is required for each different miRNA species that is detected. The small RNA species is too short to provide for primer binding sites having suitable thermal stability (Tm, or melting temperature) to support a PCR reaction without employing costly Tm increasing modifications, such as LNA bases. Primer sites therefore include both linker and miRNA specific sequences. The 5′-end of both the forward and reverse primers extend into the linker domains to whatever extent is needed to provide for sufficient Tm to support a PCR reaction using said primers. Typically the primers will vary from around 15 to 30 bases in length and will have a Tm at or around 60° C. at a primer concentration of around 200 nM in a buffer comprising around 50 mM monovalent cation (Na⁺ or K⁺) and around 3 mM divalent cation (Mg⁺⁺). The precise concentration of primer and the concentration of monovalent and divalent cations may vary, all of which will influence the Tm of the primers in ways which are well know to those with skill in the art (see Owczarzy et al., Biochemistry 47, 5336-5353 (2008); Owczarzy et al., Biochemistry 43, 3537-3554; U.S. Pat. No. 6,889,143; U.S. Patent Application 2009/0198453). Typically monovalent cation will vary from 0-50 mM and divalent cation will vary from 1.5 to 5 mM concentration. Although Mg ions are normally employed in PCR reactions, certain polymerases alternatively employ Mn cations and manganese salts can be substituted in the reaction as necessary. Once the 3′-end of a primer has been selected to maximize specificity (see below), the length of the 5′-end extending into the linker domain is varied to adjust the predicted Tm of said primer to the desired annealing temperature (typically around 60° C.).

The 3′-end of the forward and reverse PCR primers extend into the miRNA domain of the cDNA. This domain provides for the specificity of the reaction and the precise location of the 3′-end of each primer is chosen to provide for the maximum possible specificity, particularly in the context of other potential miRNA targets. Alignment of all known miRNA species (see sequence listing in miRBase, as described above) is used to define the precise position of the 3′-end of each primer, which ideally is the point where greatest sequence differences exist between the known miRNAs. The relative location of this 3′-end within the small RNA species may vary between each unique target small RNA as necessary to ensure optimal specificity. An individual primer may extend as little as 1-2 bases or as long as 15 or more bases into the miRNA domain. It is preferred that the forward and reverse primers to do not overlap. Preferably as one primer extends further into the miRNA domain the second primer will reciprocate and extends less into the miRNA domain on the opposing side. For example, for a 22 base miRNA species, a forward primer that extends 15 bases into the miRNA domain could be paired with a reverse primer that maximally extends 7 bases into the miRNA domain. Primers can be positioned so that gaps exist between forward and reverse but preferably not that in ways that result in overlap.

It is evident from examination of the hundreds of known miRNA species that careful positioning of traditional forward and reverse primers within the short body of the miRNA is inadequate to entirely ensure specificity and enable precise detection of all species from a mixture as desired. Use of blocked primers that can be cleaved by RNase H2 provides a second level of reaction specificity that enables improved discrimination of miRNA species in a qPCR reaction format.

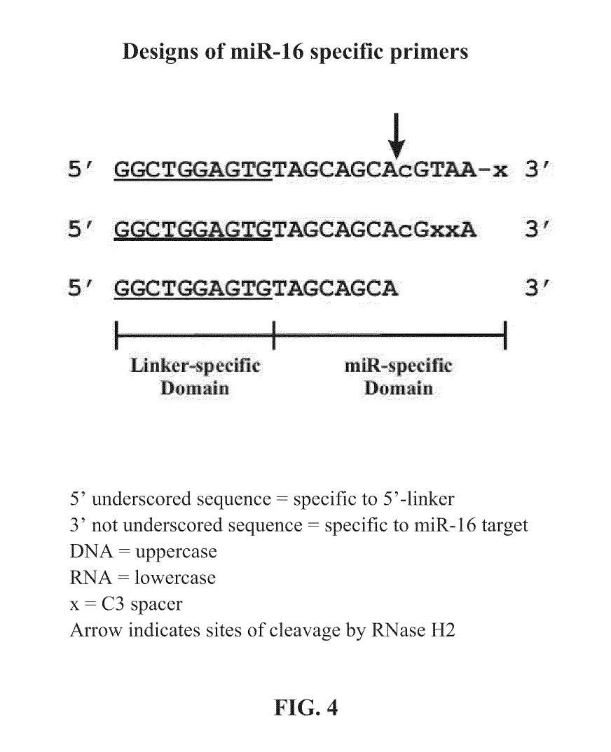

The use of RNase H2 cleavable primers to improve the specificity of PCR reactions was described by Walder et al. (U.S. Patent Application 2009/0325169) which is incorporated herein in its entirety. The primers are constructed in such a way that they are not competent to prime a DNA synthesis reaction. This can involve use of a blocking group at the 3′-end which directly prevents polymerase extension or can involve primers with an unblocked 3′-end which are otherwise modified in ways which limit their capacity to function as primers. Two variants of said primers are shown in FIG. 4 based upon miR-16 sequence, where one cleavable primer has a 3′-end block and the other cleavable primer does not but is designed such that it will not efficiently support PCR without cleavage. Another variant of a cleavable primer could involve the use of a blocking group on the 3′-end of the primer and additional modifications on the same primer. For convenience, primers of this type are referred to as blocked/cleavable primers or simply cleavable primers herein regardless of end structure or precise design.

The primers and the assay can be adapted for use in various amplification reactions known in the art. For assays involving primer extension (e.g., PCR, polynomial amplification and DNA sequencing) the modification of the oligonucleotide inhibiting activity is preferably located at or near the 3′-end. In some embodiments where the oligonucleotides are being used as primers, the oligonucleotide inhibiting activity may be positioned near the 3′ end of the oligonucleotide, e.g., up to about 10 bases from the 3′ end of the oligonucleotide of the invention.

In other embodiments, the oligonucleotide inhibiting activity may be positioned near the 3′ end, e.g., about 1-6 bases from the 3′ end of the oligonucleotide of the invention. In other embodiments, the oligonucleotide inhibiting activity may be positioned near the 3′ end, e.g., about 1-5 bases from the 3′ end of the oligonucleotide of the invention. In other embodiments, the oligonucleotide inhibiting activity may be positioned near the 3′ end, e.g., about 1-3 bases from the 3′ end of the oligonucleotide of the invention. In other embodiments, the precise position (i.e., number of bases) from the 3′ end where the oligonucleotide inhibiting activity may be positioned will depend upon factors influencing the ability of the oligonucleotide primer of the invention to hybridize to a shortened complement of itself on the target sequence (i.e., the sequence for which hybridization is desired). Such factors include but are not limited to Tm, buffer composition, and annealing temperature employed in the reaction(s).

Oligonucleotides containing a single ribonucleotide but that do not contain a fluorophore or other reporter group can be used in PCR reactions with detection by SYBR™ Green or other similar detection methods. Other embodiments provide oligonucleotides and assay formats where a reporter fluorophore and a quencher are incorporated into the cleavable primer wherein cleavage of the oligonucleotide can be measured by a change in fluorescence. In one such embodiment a primer cleavable by RNase H is labeled with a fluorophore towards one end and a quencher towards the other end and the assay is monitored by the increase in fluorescence that occurs when fluorophore and quencher are separated by the cleavage reaction.

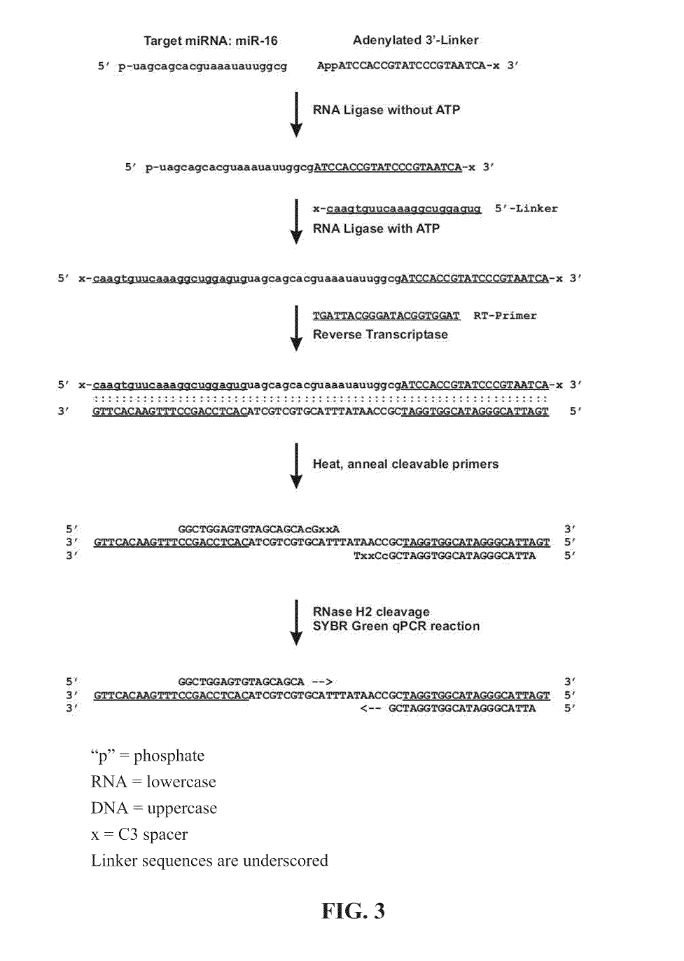

The cleavable primer assay format offers increased specificity over use of standard PCR primers. The cleavable domain provides for 5-6 additional bases at the 3′-end of each primer that hybridize to target yet which are not present in the final PCR primers. Mismatch within this domain, particularly at the ribonucleotide cleavage site, decrease or prevent primer cleavage (unblocking). Thus steps that confer specificity to the PCR reaction are present at both the cleavage/unblocking step as well as at the PCR priming step. Having two sequential, linked sequence specific events increases the overall specificity of the reaction and improves the ability of the method to distinguish between related species. Specific sequences for linkers and cleavable primers that function in detection of miR-16 using the method of the invention are described in Example 1 below and are schematically illustrated in FIG. 3. Examples of two of the many possible design variants of cleavable primers suitable for use in the method of the invention are shown in FIG. 4.

The qPCR reactions are themselves specific for individual miRNA species. Thus 600 separate reactions (for/rev primer pairs) would be required to detect 600 distinct miRNAs. The qPCR reactions can be run in high throughput in parallel fashion on 96 well plate, 384 well plate, or 1536 well plate format (etc.) or in even higher throughput formats using Fluidigm or other nanoreaction platforms, as are well known to those with skill in the art. Use of SYBR™ Green detection does not permit multiplex assays. Use of fluorescence/quenched primer formats would permit multiplex reaction formats.

The invention further provides for the use of an internal positive control which also permits assessment of the overall efficiency of the reaction process. A synthetic single-stranded RNA oligonucleotide is added to the linkering reaction (“spiked” into the RNA sample) which serves as a positive control for the linkering process. A synthetic RNA oligonucleotide “spike control” for linkering reactions was described by Devor (U.S. Patent Application 2009/0011422) in the context of a small RNA cloning process intended to identify novel miRNAs and other small RNA species by sequence analysis. The internal positive control from Devor was designed so that it would accept a 3′-linker but not a 5′-linker. It was necessary that the control not fully participate in all of the linkering and cloning reaction steps so its presence would not contaminate the final small RNA library. In the present invention, it is desired that the “spike” control be fully competent and complete all ligation steps, serve as a template for cDNA synthesis, and produce a final product that can be detected by qPCR similar to natural small RNA species. The synthetic RNA oligonucleotide structure mimics natural miRNAs, having a 5′-phosphate and 3′-hydroxyl and is preferably 21-24 bases in length. Structure and/or length of the “spike control” can be varied if small RNAs having different structure are being investigated, such as piRNAs. Such alternative structures are contemplated as part of the present invention. The sequence is selected to have no significant homology to known small RNA species and is detectably different from other known small or large RNAs in the context of the species of interest (human, mouse, rat, etc.) and so can be uniquely detected in subsequent qPCR reactions. QPCR assays specific to the synthetic “spike control” are used to detect its presence and quantify the levels present in the final mixed linkering reaction product. The “spike control” qPCR assay employs RNase H2 cleavable primers similar in design to those used to quantify natural small RNA species but are specific for the “spike” positive control RNA sequence.

A synthetic DNA oligonucleotide can be employed to generate a standard curve to permit absolute quantification. This sequence is identical to the expected cDNA product produced by successful 3′- and 5′-linkering reactions on the “spike control” RNA. Given knowledge of the input mass of the “spike control” RNA into the linkering reactions and the dilution factor used to set up the qPCR reactions, it is possible to calculate from the standard curve the amount of “spike control” cDNA present in the unknown sample and thereby estimate overall reaction efficiency. Use of the “spike control” is optional. The use is beneficial in confirming any negative results. If detection of an miRNA species of interest in the biological sample gives negative results (i.e., the qPCR reactions shows the RNA species of interest is not present), the availability of positive results from the “spike control” validates the negative result by proving that the linkering reactions were successful and even can document the efficiency of the overall reaction process.

The invention further provides for pre-designed and/or pre-validated qPCR reactions and kits to detect all known miRNA species using RNase H2 cleavable primers. In certain embodiments, the kits include a container containing an RNA ligase and another container containing an RNase H enzyme, or a single container containing an RNase H enzyme combined with an RNA ligase, and preferably there is an instruction booklet for using the kits. Optionally, the modified oligonucleotides used in the assay can be included with the enzymes. The cleavage enzyme agent, DNA polymerase and/or RNA ligase and oligonucleotides used in the assay are preferably stored in a state where they exhibit long-term stability, e.g., in suitable storage buffers or in a lyophilized or freeze dried state. In addition, the kits may further comprise a buffer for the RNase H, a buffer for the DNA polymerase or RNA ligase, or both buffers. Alternatively, the kits may further comprise a buffer suitable for the RNase H, and the DNA polymerase or RNA ligase. Buffers may include RNasin and other inhibitors of single stranded ribonucleases. Descriptions of various components of the present kits may be found in preceding sections related to various methods of the present invention.

Optionally, the kit may contain an instruction booklet providing information on how to use the kit of the present invention for amplifying or ligating nucleic acids in the presence of the novel primers and/or other novel oligonucleotides of the invention. In certain embodiments, the information includes one or more descriptions on how to use and/or store the RNase H, DNA polymerase, RNA ligase and oligonucleotides used in the assay as well as descriptions of buffer(s) for the RNase H and the DNA polymerase or RNA ligase, appropriate reaction temperature(s) and reaction time period(s), etc.

Accordingly, in one embodiment, a kit for the detection of a small RNA from a sample is provided. The kit comprises one or more of the following

(a) a 5′-adenylated synthetic oligonucleotide linker and optionally a 3′ synthetic oligonucleotide linker;
(b) an RNA ligase;
(c) a reverse transcription primer;
(d) a first and second oligonucleotide primer each having a 3′ end and 5′ end, wherein a portion of each oligonucleotide is complementary to a portion of the target miRNA and another portion of each oligonucleotide is complementary to the corresponding adjacent linker, and wherein at least one oligonucleotide comprises a RNase H cleavable domain, and a blocking group linked at or near to the 3′ end of the oligonucleotide to prevent primer extension and/or to prevent the primer from being copied by DNA synthesis directed from the opposite primer;
(e) and RNase H enzyme; and
(f) and instruction manual for amplifying the target.

In a further embodiment, the kit for selective amplification of the target includes an oligonucleotide probe having a 3′ end and a 5′ end comprising an RNase H cleavable domain, a fluorophore and a quencher, wherein the cleavable domain is positioned between the fluorophore and the quencher, and wherein the probe is complementary to a portion of the nucleic acid to be amplified or its complement.

The following examples further illustrate the invention but, of course, should not be construed as in any way limiting its scope.

### Example 1

Detection of a linkered miR-16 cDNA mimic using RNase H2 cleavable primers.

This example demonstrates the use of RNase H2 cleavable primers to detect and quantify the presence of a synthetic DNA oligonucleotide that mimics the anticipated cDNA product made from a successfully 3′- and 5′-linkering reaction from the natural miR-16 RNA sequence.

FIG. 2 is a schematic showing the sequential steps of the assay of the invention where a small RNA has linkers attached to the 3′-end, to the 5′-end, is converted to cDNA, and is detected using qPCR with RNase H2 cleavable primers. FIG. 3 shows this scheme using one possible set of linkers and employs primers specific for the detection miR-16. Note that other linker sequences could be used and the precise position of the primers within the miR-16 target could be varied. The present sequences are shown as example and are not meant to limit the scope of sequences that can be employed in the detection scheme of the invention. Cleavable primers of different designs can be employed. FIG. 4 shows two variants of RNase H2 cleavable primers as described by Walder (PCT/US09/42454) having sequences specific for detection of miR-16 in the context of the two linker sequences shown in FIG. 3. Cleavable primers are aligned with the actual functional primer species that results from cleavage by an RNase H2 enzyme. This figure is intended to show a representative example of two types of cleavable primers suitable for use with the present invention; additional designs are contemplated.

The final product of the sequential 3′-Linkering, 5′-Linker, and cDNA synthesis reactions is a 65-base single-stranded DNA species (FIG. 3). This 65-mer sequence was synthesized as a single-stranded DNA oligonucleotide for use as a positive control for testing RNase H2 cleavable primers in the context of the reaction scheme shown in FIG. 3. The synthetic oligonucleotides employed in the miR-16 detection assays of Examples 1-3 are listed below.

miR-16 For cleavable primer
SEQ ID No. 1
5′ GGCTGGAGTGTAGCAGCAcGxxA 3′
miR-16 For control primer
SEQ ID No. 2
5′ GGCTGGAGTGTAGCAGCA 3′
miR-16 Rev cleavable primer
SEQ ID No. 3
5′ ATTACGGGATACGGTGGATCGcCxxT 3′
miR-16 Rev control primer
SEQ ID No. 4
5′ ATTACGGGATACGGTGGATCG 3′
3′-adenylated DNA linker
SEQ ID No. 5
5′ AppATCCACCGTATCCCGTAATCA-x 3′
5′ RNA linker
SEQ ID No. 6
5′ x-CAAGTGuucaaaggcuggagug 3′
synthetic miR-16 amplicon
SEQ ID No. 7
5′ CAAGTGTTCAAAGGCTGGAGTGTAGCAGCACGTAAATATTGGCGA
TCCACCGTATCCCGTAATCA 3′
Where: DNA bases are uppercase
RNA bases are lowercase
x = C3 spacer.
App = 5′-adenylyl group

The following qPCR reactions were performed in SYBR™ Green detection format run on a Roche LightCycler 480 real time thermal cycler in 10 μL reactions in 384-well format. Reactions were set up as follows:

qPCR Reactions

| | | |
|---|---|---|
| BIO-RAD iQ ™ SybrGreen | 5 μL | |
| Supermix | | |
| Synthetic DNA miR-16 target | 2 μL | (2 × 10⁴ copies; SEQ ID No. 7) |
| Forward primer, 10 μM | 0.2 μL | (200 nM; SEQ ID No. 1 or 2) |
| Reverse primer, 10 μM | 0.2 μL | (200 nM; SEQ ID No. 3 or 4) |
| RNase H2 Enzyme | 1 μL | (Pyrococcus abyssi, 120 mU) |
| Water | 1.6 μL | |
| Final Volume | 10 μL | |

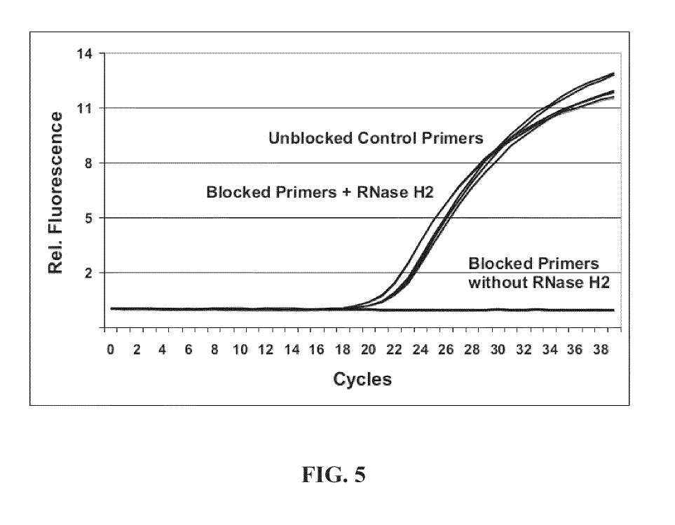

Reactions were set up using the synthetic DNA target SEQ ID No. 7 with either the unblocked control primers SEQ ID Nos. 2 and 4 or with the RNase H2 cleavable primers SEQ ID Nos. 1 and 3. All reactions were performed in triplicate. Purified Pyrococcus abyssi RNase H2 enzyme was used as taught by Walder (U.S. Application 2009/0325169). Reactions were cycled using the program 95° C. for 5 minutes followed by [95° C. for 10 seconds+60° C. for 30 seconds]×40 cycles. Results are shown in FIG. 5. As expected, the cleavable primers did not produce any signal in the absence of RNase H2 enzyme. In the presence of RNase H2, the unmodified control primers and the RNase H2 cleavable primers both showed strong fluorescence signal and were nearly indistinguishable, with a Cq (the PCR cycle when the fluorescence signal first crosses the detection threshold) of 21.8 and 22.7, respectively. Thus the RNase H2 cleavable primer assay functions well using a synthetic linkered miR16 cDNA mimic. The next example demonstrates use of the assay system to detect a synthetic miR-16 RNA, linking the detection process of Example 1 with the 3′- and 5′-linkering steps and cDNA synthesis.

### Example 2

Linkering, cDNA synthesis, and detection of a synthetic miR-16 RNA oligonucleotide using RNase H2 cleavable primers.

This example demonstrates the use of RNase H2 cleavable primers to detect and quantify the presence of a synthetic RNA oligonucleotide that mimics the functional strand of natural miR-16. In this case the synthetic RNA undergoes 3′-linkering using an adenylated DNA linker, 5′-linkering using an RNA linker, and cDNA synthesis. The final cDNA product is then detected using the RNase H2 cleavable primer qPCR assay described in Example 1 above.

In addition to the synthetic oligonucleotides described in Example 1 above, the present example employs the following synthetic RNA oligonucleotide as a miR-16 mimic

| Synthetic miR-16 RNA oligonucleotide | |
|---|---|
| 5′ p-uagcagcacguaaauauuggcg 3′ | SEQ ID No. 8 |

Step 1 of the detection method is attachment of a linker to the 3′-end of the target small RNA species, in this case the synthetic miR-16 oligonucleotide. The following reaction mix was prepared. After final assembly and dilution, the composition of 1X RNA Ligation buffer is 33 mM Tris acetate pH 7.8, 66 mM potassium acetate, 10 mM magnesium acetate, and 0.5 mM dithiothreitol (DTT). DMSO is added to a final concentration of 30%, which serves to enhance the efficiency of single-stranded RNA ligation reactions (see Devor et al., U.S. Application 2009/0011422). The T4 RNA Ligase was from Epicentre (Madison, Wis., USA; Catalog #LR5010, 5 U/μL). Note that the use of excess RNA Ligase can lead to lower linkering efficiency and the precise amount of RNA Ligase employed may need to be titrated for different input RNA target mixtures, a process well known to those with skill in the art. Stock solutions of the synthetic miR-16 RNA (SEQ ID No. 8) was at 5 μM concentration and the Adenylated 3′-linker (SEQ ID No. 5) was at 5 μM concentration.

3′-Linkering Reaction

| | | |
|---|---|---|
| 10× Ligation Buffer | 2 μL | (final 1X) |
| miR-16 RNA, 5 μM | 5 μL | (25 pmole = 1.25 μM; SEQ ID No. 8) |
| Adenylated 3′-Linker, | 5 μL | (5 pmole = 1.25 μM; SEQ ID No. 5) |
| 5 μM | | |
| DMSO ligation enhancer | 6 μL | (final = 30%) |
| T4 RNA Ligase | 1 μL | (diluted 5x, final = 1 unit) |
| Water | 1 μL | |
| Final volume | 20 μL | |

A negative control reaction was set up and run in parallel without any input RNA. Additional water was added (5 μL) to maintain a final 20 μL reaction volume.

The 3′-linkering reactions were incubated at 22° C. for 2 hours using T4 RNA Ligase in the absence of ATP, after which the 5′-linkering reactions were set up by direct addition of the following components to the above reaction mixture (Step 2 of the detection method). The stock solution of 5′-Linker was at 25 μM concentration and stock solution of ATP was at 25 mM concentration.

5′-Linkering Reaction

| | | |
|---|---|---|
| 3′-linkering reaction | 20 μL | (from above) |
| T4 RNA Ligase | 1 μL | (5 units) |
| 5′-Linker, 25 μM | 2 μL | (final 50 pmole = 2 μM; SEQ ID No. 6) |
| ATP, 25 mM | 1 μL | (final 1 mM) |
| Water | 1 μL | |
| Final volume | 25 μL | |

The 5′-linkering reactions were incubated at 22° C. for 2 hours using T4 RNA Ligase in the presence of ATP. The reactions were stopped by heating at 65° C. for 10 minutes to inactivate the ligase enzyme.

Step 3 of the detection method is conversion of the linkered small RNA species to cDNA by reverse transcription. The RT reactions employed the Superscript-III reverse transcriptase enzyme (Invitrogen, Carlsbad, Calif.; Cat # 18080-051, 200 U/μL). Composition of 1X RT Buffer is 50 mM Tris-HCl pH 8.3, 75 mM KCl, 3 mM MgCl₂, and 5 mM DTT. The primer used for reverse transcription was a DNA oligonucleotide complementary to the adenylated 3′-Linker and was at 10 μM stock concentration. SUPERase-IN was included in the reaction mix and is a peptide inhibitor of single-strand RNases (Ambion, Austin, Tex.; Cat #AM2696). 1 μL of the above linkering reactions were used as template in the reverse transcription reactions.

| RT Primer | |
|---|---|
| 5′ TGATTACGGGATACGGTGGAT 3′ | SEQ ID No. 9 |

Reverse Transcription Reactions

| | | |
|---|---|---|
| Linkering Reaction (LR) | 1 μL | (from above) |
| 5X RT Buffer | 4 μL | (final 1X) |
| 10 mM dNTPs | 1 μL | (final 0.5 mM) |

This mixture was heated at 65° C. for 5 minutes and quick chilled on ice. Next, the final reaction components were added:

| | | |
|---|---|---|
| RT Primer, 10 μM | 1 μL | (final 500 nM; SEQ ID No. 9) |
| 0.1M DTT | 2 μL | (final 10 mM) |
| SUPERase-IN | 1 μL | (final 20 units) |
| Superscript-III RT enzyme | 1 μL | (final 200 units) |
| Water | 9 μL | |
| Final volume | 20 μL | |

The reverse transcription reactions were incubated at 50° C. for 1 hour and stopped by incubation at 70° C. for 15 minutes. Serial dilutions were made from the cDNA synthesis reactions for use in qPCR reactions to detect the presence of linkered miR-16 species. Dilutions were made at 10⁻³, 10⁻⁴, 10⁻⁵, and 10⁻⁶.

Step 4 of the detection method is qPCR detection and quantification of the cDNA product derived from the input small RNA species. The “minus RNA linkering/cDNA reaction served as negative control. The following qPCR reactions were performed in SYBR™ Green detection format run on a Roche LightCycler® 480 real time thermal cycler in 10 μL reactions in 384-well format. Reactions were set up as follows:

qPCR Reactions

| | | |
|---|---|---|
| BIO-RAD iQ ™ SYBR Green | 5 μL | |
| Supermix | | |
| cDNA product, various dilutions | 1 μL | (from above) |
| Forward primer, 10 μM | 0.2 μL | (200 nM; SEQ ID No. 1) |
| Reverse primer, 10 μM | 0.2 μL | (200 nM; SEQ ID No. 3) |
| RNase H2 Enzyme | 1 μL | (Pyrococcus abyssi, 120 mU) |
| Water | 2.6 μL | |
| Final Volume | 10 μL | |

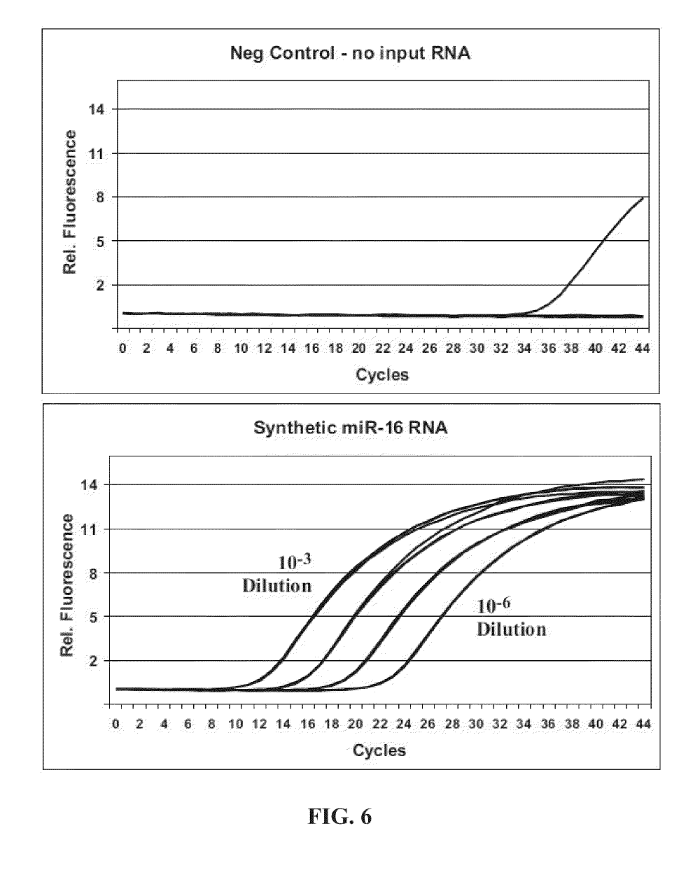

Reactions were set up using the RNase H2 cleavable primers SEQ ID Nos. 1 and 3. All reactions were performed in triplicate. Purified Pyrococcus abyssi RNase H2 enzyme was used. Reactions were cycled using the program 95° C. for 5 minutes followed by [95° C. for 10 seconds+60° C. for 30 seconds]×45 cycles. Results are shown in FIG. 6. The amplification reactions using the RNase H2 cleavable primers showed strong fluorescence signals with average Cq's (the PCR cycle when the fluorescence signal first crosses the detection threshold) of 13.1 (10⁻³ dilution), 16.6 (10⁻⁴ dilution), 20.1 (10⁻⁵ dilution), and 23.7 (10⁻⁶ dilution), varying as expected with the dilution factor of the input cDNA target. All reactions were run in triplicate, so each curve represents an overlay of 3 real time PCR reactions. The negative control reaction (run in parallel, including 3′-linkering, 5′-linkering, cDNA synthesis, and qPCR reactions) was included to define any background signal present that may arise from linker dimers or other nucleic acid interactions; the same set of 4 dilutions were similarly run in triplicate for the control. The negative control reactions showed no amplification in 11/12 assays; a single reaction from the 10⁻⁶ dilution set showed positive signal at 36 cycles and presumably represents low level contamination and not a true false positive signal.

Given the known input of synthetic miR-16 RNA (25 pmole), these values represent an overall efficiency of ˜20% for the combined series of linkering, cDNA conversion, and detection reactions. It is expected that using higher molar ratios of linker to target will result in efficiencies closer to 100%.

Thus the RNase H2 cleavable primer assay functions well using a synthetic miR16RNA species. The next example demonstrates use of the assay system to detect a natural miR-16 RNA within a heterogeneous total RNA mixture purified from human cells in tissue culture, linking the detection process of Example 1 with the 3′- and 5′-linkering steps and cDNA synthesis steps of Example 2.

### Example 3

Linkering, cDNA synthesis, and detection of natural miR-16 from purified total cellular RNA using RNase H2 cleavable primers.

This example demonstrates use of RNase H2 cleavable primers to detect and quantify the presence of miR-16 in a natural RNA sample prepared from cultured human cells. In this case an unfractionated total RNA population undergoes 3′-linkering using an adenylated DNA linker, 5′-linkering using an RNA linker, and cDNA synthesis using the method described in Example 2 above (FIGS. 2 and 3). The final cDNA product is then detected using the RNase H2 cleavable primer qPCR assay described in Example 1 above (FIG. 1).

Step 1 of the detection method is attachment of a linker to the 3′-end of the target small RNA species, in this case natural miR-16 and other species present in total HeLa cell RNA. Total RNA was prepared from HeLa cells using RNA STAT-60™ (Tel-Test, Friendswood, Tex.) using the manufacturer's protocols. Organic liquid extraction of cellular RNA is generally the preferred method to isolate RNA for detection of small RNA species. Most solid phase resin binding RNA isolation methods lose small RNA species which is undesirable for the present application.

The following reaction mix was prepared. After final assembly and dilution, the composition of 1X RNA Ligation buffer is 33 mM Tris acetate pH 7.8, 66 mM potassium acetate, 10 mM magnesium acetate, and 0.5 mM dithiothreitol (DTT). DMSO is added to a final concentration of 30%, which serves to enhance the efficiency of single-stranded RNA ligation reactions (see Devor et al., U.S. Patent Application 2009/0011422). The T4 RNA Ligase was from Epicentre (Madison, Wis., USA; Catalog #LR5010, 5 U/μL). Note that the use of excess RNA Ligase can lead to lower linkering efficiency and the precise amount of RNA Ligase employed may need to be titrated for different input RNA target mixtures, a process well known to those with skill in the art. Stock solution of the HeLa cell total RNA was at 20 ng/μL concentration and the adenylated 3′-linker (SEQ ID No. 5) was at 5 μM concentration.

3′-Linerking Reaction

| | | |
|---|---|---|
| 10X Ligation Buffer | 2 μL | (final 1X) |
| HeLa RNA, 20 ng/μL | 5 μL | (100 ng) |
| Adenylated 3′-Linker, 5 μM | 1 μL | (4.5 pmole = 30 ng = 0.25 μM; |
| | | SEQ ID No. 5) |
| DMSO ligation enhancer | 6 μL | (final = 30%) |
| T4 RNA Ligase | 1 μL | (diluted 5x, final = 1 unit) |
| Water | 5 μL | |
| Final volume | 20 μL | |

A negative control reaction was set up and run in parallel without any input RNA. Additional water was added (5 μL) to maintain a final 20 μL reaction volume.

The 3′-linkering reactions were incubated at 22° C. for 2 hours using T4 RNA Ligase in the absence of ATP, after which the 5′-linkering reactions were set up by direct addition of the following components to the above reaction mixture (Step 2 of the detection method). The stock solution of 5′-Linker was at 25 μM concentration and stock solution of ATP was at 25 mM concentration.

5′-Linkering Reaction

| | | |
|---|---|---|
| 3′-linkering reaction | 20 μL | (from above) |
| T4 RNA Ligase | 1 μL | (5 units) |
| 5′-Linker, 25 μM | 2 μL | (final 50 pmole = 2 μM; SEQ ID No. 6) |
| ATP, 25 mM | 1 μL | (final 1 mM) |
| Water | 1 μL | |
| Final volume | 25 μL | |

The 5′-linkering reactions were incubated at 22° C. for 2 hours using T4 RNA Ligase in the presence of ATP. The reactions were stopped by heating at 65° C. for 10 minutes to inactivate the ligase enzyme.

Step 3 of the detection method is conversion of the linkered small RNA species to cDNA by reverse transcription. The RT reactions employed the Superscript-III reverse transcriptase enzyme (Invitrogen, Carlsbad, Calif.; Cat # 18080-051, 200 U/μL). Composition of 1X RT Buffer is 50 mM Tris-HCl pH 8.3, 75 mM KCl, 3 mM MgCl₂, and 5 mM DTT. The primer used for reverse transcription was a DNA oligonucleotide complementary to the adenylated 3′-Linker and was at 10 μM stock concentration. SUPERase-IN was included in the reaction mix and is a peptide inhibitor of single-strand RNases (Ambion, Austin, Tex.; Cat #AM2696). 1 μL of the above linkering reaction was used in the reverse transcription reaction.

Reverse Transcription Reaction

| | | |
|---|---|---|
| Linkering Reaction (LR) | 1 μL | (from above) |
| 5X RT Buffer | 4 μL | (final 1X) |
| 10 mM dNTPs | 1 μL | (final 0.5 mM) |

These mixtures were heated at 65° C. for 5 minutes and quick chilled on ice. Next, the final reaction components were added:

| | | |
|---|---|---|
| RT Primer, 10 μM | 1 μL | (final 500 nM; SEQ ID No. 9) |
| 0.1M DTT | 2 μL | (final 10 mM) |
| SUPERase-IN | 1 μL | (final 20 units) |
| Superscript-III RT enzyme | 1 μL | (final 200 units) |
| Water | 9 μL | |
| Final volume | 20 μL | |

The reverse transcription reactions were incubated at 50° C. for 1 hour and stopped by incubation at 70° C. for 15 minutes.

Step 4 of the detection method is qPCR detection and quantification of the cDNA product derived from the input small RNA species. The following qPCR reactions were performed in SYBR™ Green detection format run on a Roche LightCycler® 480 real time thermal cycler in 10 μL reactions in 384-well format. Reactions were set up as follows:

qPCR Reactions

| | | |
|---|---|---|
| BIO-RAD iQ ™ SYBR Green | 5 μL | |
| Supermix | | |
| cDNA product, undiluted | 1 μL | (from above) |
| Forward primer, 10 μM | 0.2 μL | (200 nM; SEQ ID No. 1) |
| Reverse primer, 10 μM | 0.2 μL | (200 nM; SEQ ID No. 3) |
| RNase H2 Enzyme | 1 μL | (Pyrococcus abyssi, 120 mU) |
| Water | 2.6 μL | |
| Final Volume | 10 μL | |

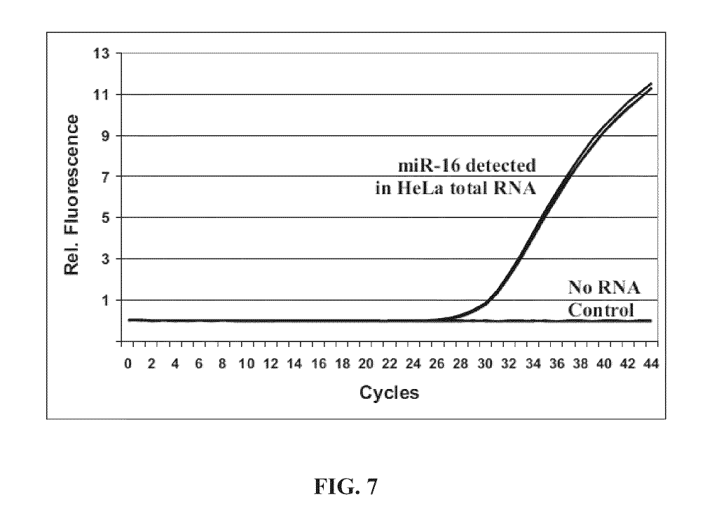

Reactions were set up using the RNase H2 cleavable primers SEQ ID Nos. 1 and 3. All reactions were performed in triplicate. 1 μL of undiluted cDNA synthesis reaction product (for both the HeLa cell and no RNA negative control samples) was used for each qPCR reaction. For the HeLa RNA samples, the input cDNA into these reactions corresponds to the equivalent of 0.4 ng of the starting total cellular RNA used in the initial linkering reaction. Purified Pyrococcus abyssi RNase H2 enzyme was used. Reactions were cycled using the program 95° C. for 5 minutes followed by [95° C. for 10 seconds+60° C. for 30 seconds]×45 cycles. Results are shown in FIG. 7. The RNase H2 cleavable primers showed strong fluorescence signal with a Cq (the PCR cycle when the fluorescence signal first crosses the detection threshold) of 31.0. No signal was detected in the “no RNA” negative control reactions, confirming that linker products alone did not produce a false positive signal. Thus the RNase H2 cleavable primer assay functions well using to detect miR-16 species present in heterogeneous sample of total RNA isolated from human cells.

### Example 4

The following example demonstrates methods for computationally optimizing sequences for the 3′-Linker, the 5′-Linker, and a synthetic miRNA Spike Control RNA species.

FIG. 3 and Examples 2 and 3 demonstrate linkering and detection of small RNA species using two specific linker sequences, SEQ ID Nos. 5 and 6. These linkers functioned well in the detection of miR-16 starting with either a synthetic miR-16 oligonucleotide or total HeLa cellular RNA. These sequences were selected because both have been previously used as universal tags and were known to support PCR reactions. The sequences did not have any palindromic domains and did not form stable hairpin, self-dimer or heterodimer structure. However, these linker sequences were not specifically optimized for use in the context of small RNA detection. New candidate sequences for synthetic 3′-linker, 5′-linker, and positive control spike species using computational methods intended to be maximally unique from any known small RNA species (miRBase, http://www.mirbase.org/) were derived. A sequence length of 22 bases was selected for the derivation process with this length being representative of an average miRNA and also being a suitable length for a primer domain.

To exhaustively search the space of possible 22mers to find those that are maximally distant in edit distance from the sequences contained in miRBase is an intractable problem, considering that the number of unique 22-mer sequences possible is approximately 1.76×10¹³ (i.e., 4²²). To avoid the intractability of an exhaustive search, a randomization method was employed. A population of random 22mers was generated from a uniform base distribution under the following conditions: runs of length 4 or more of a single base were prohibited, consecutive doublet or triplet repeats of length 6 or more (for example CTCTCT or ACGACG) were prohibited, and repeats of length 4 or more anywhere in the sequence (not necessarily consecutively) were prohibited. Once this population was generated, the edit distance of each possible sequence was calculated against each sequence in the mature and mature* miRBase sequences listed obtained from miRBase release 14 (September 2009). For each generated sequence, the minimum edit distance to a sequence in miRBase is recorded. Once the minimum edit distances are collected for the entire generated population, those with the largest minima are collected to be considered for final use as linkers and controls for the assay system. Sequences that are maximally distant from both miRBase and from each other were selected. For a group of three sequences, a mutual distance vector of length three can be constructed where each entry is the edit distance between one pair in the group of three. This mutual distance vector can be calculated for every group of three in the set of sequences maximally distant from miRBase entries. Those groups of 3 with mutual distance vectors having the largest Euclidean norms will most likely be best suited for use as a linker/control set for the assay system.

Using this computational method, a set of 321 sequences were defined that were at least an edit distance of 9 from each other and from any entry in miRBase. These sequences are listed in Table 1. Many additional sequences are likely to be suitable choices for use as linkers or controls, and the use of such additional sequences for this application is contemplated within the invention. The sequences defined herein are one set having suitable sequence features for use in the method of the invention and are not meant to be limit scope.

TABLE 1
Sample defined sequences having sequence
features compatible with miRNA detection assay

| SEQ ID No. | Sequence |
|---|---|
| SEQ ID No. 10 | GGUUUCGGAGAACCCGUGGGCU |
| SEQ ID No. 11 | GAGUAAAUUCGCUCAACUACGU |
| SEQ ID No. 12 | ACUUUCCGGUAGGAUUAACCAA |
| SEQ ID No. 13 | CCGAAUUUAUCCUCGGCGAUUG |
| SEQ ID No. 14 | CUUAUCUACAAGUACGCGAUAA |
| SEQ ID No. 15 | GCUGACGUAUAAAUGCCCUCGA |
| SEQ ID No. 16 | CAGCAAGAGGCGACCCAAAGGU |
| SEQ ID No. 17 | GAACGUUGGAUACGCCCGUGUA |
| SEQ ID No. 18 | CCGCCUCCAUUUCAACCUAGAA |
| SEQ ID No. 19 | CCCGCGCCUUCGGAUGCAUGGG |
| SEQ ID No. 20 | GACUCUAAGUGAGCCAGGGUCG |
| SEQ ID No. 21 | AUAACCAUAUCGCCUCCCUGGG |
| SEQ ID No. 22 | GCCCGAGCUCUACCGCGUCAAA |
| SEQ ID No. 23 | GCCGUGUUACUAAACUCCUUGG |
| SEQ ID No. 24 | GUAUGGCCAAAUUAGCCUCGAA |
| SEQ ID No. 25 | CAAUUAAGGAGUCAUUCACGCC |
| SEQ ID No. 26 | CCUAUAUUCCGAUCGAGCACGA |
| SEQ ID No. 27 | GGCAGCUUAGUUAAUCUAUAAA |
| SEQ ID No. 28 | GACUUUGCCUCAAGGGACGAUU |
| SEQ ID No. 29 | GCGCUACAGACAUGCGGCCCAA |
| SEQ ID No. 30 | GCCGUCCGGGAUUCUUAAAUUA |
| SEQ ID No. 31 | CCAUCUCUACGACACGUAGGAA |
| SEQ ID No. 32 | GGCGCAUAAGAAUCCAACGAUU |
| SEQ ID No. 33 | GUGUUUAACUUUGGCUACGCCC |
| SEQ ID No. 34 | GGCGCGACCCUUUAAACUAUCC |
| SEQ ID No. 35 | CCAAGGGCCGAAUCAUUCCGUA |
| SEQ ID No. 36 | AAUAUCGCGUAACCCGCUCUAG |
| SEQ ID No. 37 | UGGUUAAGGGCUUCCCGAAAGC |
| SEQ ID No. 38 | ACCUUUCUCGUACACGCGCAUA |
| SEQ ID No. 39 | GGCGUGUGGAAUCCGCCAACCG |
| SEQ ID No. 40 | GAGAAAUCUUACACAGGCCAUC |
| SEQ ID No. 41 | GGACCCUUCCGAAACUUUAUAA |
| SEQ ID No. 42 | GGGAGUAUCGCCCAUUACUUUA |
| SEQ ID No. 43 | GAGAACAGGGCCUAUGAUAUCC |
| SEQ ID No. 44 | AUUCUACCACAAUGUCGCGUUA |
| SEQ ID No. 45 | GCUCGAACGGCCAAGACAUAUG |
| SEQ ID No. 46 | ACCCAAGCUAACCGAUACGCCC |
| SEQ ID No. 47 | CCGACGUUUAGCCCAACCUCCC |
| SEQ ID No. 48 | ACCUUAGGUUUACCGGGCCGAC |
| SEQ ID No. 49 | GCCGUUCCAGGACGCACUAAAG |
| SEQ ID No. 50 | CCCUCCGGGUUUGUCAAUAAGG |
| SEQ ID No. 51 | GAUACGUUCCGAACAAUAACCG |
| SEQ ID No. 52 | GCAAUACUCAUCUUAAAUCCUA |
| SEQ ID No. 53 | CCGAUUCUGCUCUUUGAAAGGG |
| SEQ ID No. 54 | CCGAUCCGGCGAGCGUCAACAU |
| SEQ ID No. 55 | GCUUUAGGGCUACGUAUAGACC |
| SEQ ID No. 56 | AAGGGCUCCUAAGCAACCCGUC |
| SEQ ID No. 57 | CUGCCAGUGGGUACGGCAAAUG |
| SEQ ID No. 58 | GGGAAGGGUGAUUCCUUAAAUG |
| SEQ ID No. 59 | GACCAGACGCCCUCGCGUCAAU |
| SEQ ID No. 60 | GAAGCAACCUCAAUUUACACUG |
| SEQ ID No. 61 | CCCUAAGCGUCCUUCCGAAAUA |
| SEQ ID No. 62 | AUUGCGGGCUGCCGGUCCAAAG |
| SEQ ID No. 63 | ACAUACACGGUCCUUAUGCGUA |
| SEQ ID No. 64 | AACAAACGGGAUUUGAUAUACG |
| SEQ ID No. 65 | GGUUUCGCUUACUAUAACCAAU |
| SEQ ID No. 66 | GCGUUUGCACGCUUAUAAAUUA |
| SEQ ID No. 67 | GUUCCAAAUAUUUAGGCCUAAU |
| SEQ ID No. 68 | ACGGCGACGUAAACGAACCCUC |
| SEQ ID No. 69 | GUAGACGAAAUCAAGCCACCGC |
| SEQ ID No. 70 | CCUUUGCUGUAACUUAAGCGCC |
| SEQ ID No. 71 | GGCCCUAUGGAGUGUGCCAAUU |
| SEQ ID No. 72 | AUAACGCUUGCGCCGAUCAACU |
| SEQ ID No. 73 | GUCGCUACGGUAACCCAGUCAC |
| SEQ ID No. 74 | GUAUGGGCCUCGAAAGGGAUCG |
| SEQ ID No. 75 | ACCCGUCGACUUACGGACAAAU |
| SEQ ID No. 76 | ACUACGCUCCGGAUAUUAGACC |
| SEQ ID No. 77 | CACCGUCAAGGUUAUAAACAAA |
| SEQ ID No. 78 | GGCAUACAACGACCCUAGUGAU |
| SEQ ID No. 79 | GGGAUGCGGUCUCCUAAACGCG |
| SEQ ID No. 80 | GCUUAUACCGUUGAAUAAACGU |
| SEQ ID No. 81 | GUUACGAUUGGUCCAUAACAGA |
| SEQ ID No. 82 | CCGCGCAAUGGGUGGAUACAAG |
| SEQ ID No. 83 | GCGGAACAAUUAUCUUAGAGCC |
| SEQ ID No. 84 | CCCGCCAAUAACCUCGAAGGAA |
| SEQ ID No. 85 | CUCGCGUUAUACCAGAAGGAGU |
| SEQ ID No. 86 | AAGAAUAAACGGCCCAUCGCUA |
| SEQ ID No. 87 | CGCAAUUAGACCAGUUAACGGG |
| SEQ ID No. 88 | CGUAUACAGCGGCCGCUCAACG |
| SEQ ID No. 89 | GCCCAACAUCCGGUCAGGUAAA |
| SEQ ID No. 90 | GGUGUCCGGAUAAUUCAAACUA |
| SEQ ID No. 91 | CGCCGGCGAAGCUUAACAGAAA |
| SEQ ID No. 92 | GGCGUAAUACAAUCUCCAUGCC |
| SEQ ID No. 93 | GAUAUAAUCAGAUCCCGGGUGG |
| SEQ ID No. 94 | CCACUAUCCAAUCAACGUUAGG |
| SEQ ID No. 95 | CCGACUCUAAGAGGCACAAGGU |
| SEQ ID No. 96 | GGCCUUUAGUGAACCGCGCAUC |
| SEQ ID No. 97 | CAACCAAUAUCAUAAGAUCGUC |
| SEQ ID No. 98 | GCCCGGAAAUAAGACCAAGUGG |
| SEQ ID No. 99 | CGUCGAACUCCAAUUAAAGAUC |
| SEQ ID No. 100 | GCCUUUAGGCUACCCUCAAUUA |
| SEQ ID No. 101 | GUACACUACGGGCCCACGACCA |
| SEQ ID No. 102 | GUCACAAUUUAAACCAGAUUCG |
| SEQ ID No. 103 | GUGUCGUUACUCACCUAAUUUG |
| SEQ ID No. 104 | GACUUGCCUUAACCCUAUACUG |
| SEQ ID No. 105 | AAUAACAUAGGGCCAUUCUUAA |
| SEQ ID No. 106 | GGUUACAUACCGCCCUAGCGUU |
| SEQ ID No. 107 | GGUGUUUACGGCUAAACGUAAG |
| SEQ ID No. 108 | GAGCGAGUUCUUUCGACGGGCC |
| SEQ ID No. 109 | GAAACGGAACAAUCCACCUUCU |
| SEQ ID No. 110 | UUUCCGGUAGUGACCCUACGUA |
| SEQ ID No. 111 | CGUACGGACGCUCAGAAUCAAC |
| SEQ ID No. 112 | CGCCAACCUCGGCUAAUCGUAA |
| SEQ ID No. 113 | GGAAGAGGUGUUCCCGCGGCAC |
| SEQ ID No. 114 | GGCCCUUCAUACUCGUGAAACG |
| SEQ ID No. 115 | UGAUCCAAGCGCUUAUGCAAUA |
| SEQ ID No. 116 | GAAACUGAUUAGAUACCCGCCC |
| SEQ ID No. 117 | CCAAGGCGACGUCCAUAUCCGG |
| SEQ ID No. 118 | CCGCGCUUUCGAGGGCCCGUAU |
| SEQ ID No. 119 | GAACACCUAACGGCGUGGUAGG |
| SEQ ID No. 120 | AAACGGAGAACCACUAGCUAAG |
| SEQ ID No. 121 | UGUUAUAUCAAGCCCUAGGGAA |
| SEQ ID No. 122 | GUUCAGCGCGAACGCAAUCCGA |
| SEQ ID No. 123 | GACUCAUCGCAAUAGGCGGGAA |
| SEQ ID No. 124 | GGGUGGAUUGGGACCCUGCCCG |
| SEQ ID No. 125 | CCGUCGAGAGGAUGGUUCUUUA |
| SEQ ID No. 126 | AUUUGGCCCUAACUAUAUCCGG |
| SEQ ID No. 127 | CCCUAGGUAUCCGGCGCAAGUA |
| SEQ ID No. 128 | CCGGUCCACAAUCCCAGAGAUA |
| SEQ ID No. 129 | GGUUUAACAUACGCGAAGCUAA |
| SEQ ID No. 130 | GAAAUCCCAUACUAUUCGCCCG |
| SEQ ID No. 131 | CUCGCGACCAAAUCCGUUUAAA |
| SEQ ID No. 132 | CCCAUGCUUACGAUAGGCGAAA |
| SEQ ID No. 133 | GGCUUGCCCACAGAUUUAAAGG |
| SEQ ID No. 134 | GCCGGCGAACCUAUCAACGUUA |
| SEQ ID No. 135 | GACGCGAAUAACUUCAUCUCUA |
| SEQ ID No. 136 | AGACGCCUCAACUGUUACCCGA |
| SEQ ID No. 137 | GGUCCUUACGUAGGUUGGGAAA |
| SEQ ID No. 138 | GGCAACUCCGCAGCUUAUUUAC |
| SEQ ID No. 139 | GGCCCAAACCGGAAUCGAGGGA |
| SEQ ID No. 140 | GGCCCGCGAACGUACCUAAAUA |
| SEQ ID No. 141 | CUACGAGCAUUUAGCCCGUCAC |
| SEQ ID No. 142 | GAGUUCGUCUAAGAUGAGACCC |
| SEQ ID No. 143 | CGGAUUGGGUUCCAACCACAAA |
| SEQ ID No. 144 | GACACGUCGCGCAACUAAAGAG |
| SEQ ID No. 145 | GCUCGCGUAGACACUAUCCGAU |
| SEQ ID No. 146 | CUGCCGGCCUAUUCAAGUAAAG |
| SEQ ID No. 147 | GUUUGAAACCGGACAGGUUCCU |
| SEQ ID No. 148 | AUCCCGACGGCGAAAGCUAUCG |
| SEQ ID No. 149 | CAAGACGUCACGAACCGAGCAA |
| SEQ ID No. 150 | GGCACCGUCGGAUCACGAAUAU |
| SEQ ID No. 151 | GAUCAGUUUCACCCGAACCAAA |
| SEQ ID No. 152 | GACGCUCGGUAAGUUAACCAAG |
| SEQ ID No. 153 | UUAAUCGGGUUCCCGCGUGGCU |
| SEQ ID No. 154 | CCUACGAGUACUUAACCCGUCC |
| SEQ ID No. 155 | CAUACGCGAUCUGCCCAAACCU |
| SEQ ID No. 156 | AAACGGUUGAGCUACGCUUAUA |
| SEQ ID No. 157 | CAAUUCCCGCCAGCGCGUGGGU |
| SEQ ID No. 158 | GUGGCUCAUACCGGUCAAUCCC |
| SEQ ID No. 159 | CACGAUAGUUAAGCAACCCGCC |
| SEQ ID No. 160 | GGACCGUUAAACGGUGUGCGCG |
| SEQ ID No. 161 | CCGGAGCAUUGGCCACGAGUGC |
| SEQ ID No. 162 | CGAGGCUCCCUCAACGGUAAAG |
| SEQ ID No. 163 | CCUGGUGUAUGUUUCGACACCG |
| SEQ ID No. 164 | CCGCCCGGAAUGUACAAUAAAC |
| SEQ ID No. 165 | GGCGAUAAACGUGGCCCUUAGG |
| SEQ ID No. 166 | GCGGGAACCUAUAGACUCAGGG |
| SEQ ID No. 167 | GCCUUAAUGGCGUUCGCGCCGU |
| SEQ ID No. 168 | GACUUACAGACGCGCCGAGAAA |
| SEQ ID No. 169 | CCGCAAGCUUUGGGAAUUAUAA |
| SEQ ID No. 170 | GACGCUGCCCUCUACAUCGCGA |
| SEQ ID No. 171 | CCGCGCCAAACCAUCGAAGUUG |
| SEQ ID No. 172 | GAAUCCCGCCCACUAUCGCGAC |
| SEQ ID No. 173 | GGUCAACCGGAUUACUAUGGUA |
| SEQ ID No. 174 | GGGAUGAGAAGUUCGCACCCAA |
| SEQ ID No. 175 | GAUAUUGCCAGCUCCGGUUAAC |
| SEQ ID No. 176 | CGCAUUAUCUAGAACCGUCAGA |
| SEQ ID No. 177 | GUCACAGAAUCGACCUUAAAGU |
| SEQ ID No. 178 | CACGGUAAUCAGCCGACGCAAU |
| SEQ ID No. 179 | CCGCCCUAAAUCUGGGCCAAUG |
| SEQ ID No. 180 | AGGUAAUAGCACUAACCCGCCC |
| SEQ ID No. 181 | GGCUCCUUAUCCGUAGCGGAAA |
| SEQ ID No. 182 | ACUUACUCAAUAGCCUUUAGAG |
| SEQ ID No. 183 | GUAUAAUUGCAACCGCCUCGAC |
| SEQ ID No. 184 | CCUACCCAAAGGCGGACGCGAC |
| SEQ ID No. 185 | ACCGUCGGACCAUGCGUAAUAC |
| SEQ ID No. 186 | CGCGGGAAAGUACCGGACUAAA |
| SEQ ID No. 187 | CCCUCGCUCUACGUUAAGGCUG |
| SEQ ID No. 188 | AUUAACCCAGACAUCUUCGAGC |
| SEQ ID No. 189 | CUGCCGUUCCGGGUUGAGCAAA |
| SEQ ID No. 190 | CAAAUCCGGUACCCUUAAACUA |
| SEQ ID No. 191 | CGGUCUGCUUUCGGAAUAAAGC |
| SEQ ID No. 192 | GAAGGGAAUAUCUCGGACAACC |
| SEQ ID No. 193 | AAUCGGACAGCGCAAACGGCCC |
| SEQ ID No. 194 | GGGAUACCCAAGAAAUAGUCCC |
| SEQ ID No. 195 | GGUACCACGAAAUCAAGGCACA |
| SEQ ID No. 196 | CUCGGCCCACGCAGUUUAAGGU |
| SEQ ID No. 197 | GACCGAAACAGUCGUGUUGACG |
| SEQ ID No. 198 | GGUAGGGCGUCCGCCCUAACUC |
| SEQ ID No. 199 | GCAGUAAAGCCGUAUAAGGCCA |
| SEQ ID No. 200 | GUCAAUUUCUCUUACCCGAACA |
| SEQ ID No. 201 | GCGAAAUGGCAUACCACUCCCA |
| SEQ ID No. 202 | CCAUAGCUUUGCGGCCAACUAG |
| SEQ ID No. 203 | CCUAGCCAGAAUCCGGCGUUCG |
| SEQ ID No. 204 | ACGAACGGCUAACCUACCAUAA |
| SEQ ID No. 205 | GCUCCGGAUCAACGUGGCGAAG |
| SEQ ID No. 206 | AAACGAAUCCCUCAUUUAUACU |
| SEQ ID No. 207 | CGCUUAGUAAUGACCUCACGCC |
| SEQ ID No. 208 | GCCCAUGUUAAGCGUAAAUAGA |
| SEQ ID No. 209 | GAUAUUGCCGUCGCCUAGGUCC |
| SEQ ID No. 210 | AUGAAGUCCCUAAUACCAACCC |
| SEQ ID No. 211 | CCUGAUGUUUAGCGCUCAGGUU |
| SEQ ID No. 212 | GGGAUACACGUUCCAACCGGAA |
| SEQ ID No. 213 | GGCACUUUACGUCCUUGGCCGA |
| SEQ ID No. 214 | GGGAACGUCCGGCAACUUAAAU |
| SEQ ID No. 215 | CCCGCUCCCUUAACUGCGAAUU |
| SEQ ID No. 216 | CGGUCCGCGGCAUGCCACGAAA |
| SEQ ID No. 217 | GGUGAAGGCCAAACCCUGCAAU |
| SEQ ID No. 218 | GCCAUAUGGCUCCCGGAAAUCG |
| SEQ ID No. 219 | CGUGCCCAGAUAAUGGAUUUAG |
| SEQ ID No. 220 | GGUUCAAACGCGUACCUGGCGG |
| SEQ ID No. 221 | GACAAAUUCCCGAGGGUUCGCG |
| SEQ ID No. 222 | ACAACUACGCGGACCGAAGCAA |
| SEQ ID No. 223 | CCGCCAUUAAGGCUACGCAAAU |
| SEQ ID No. 224 | GGAUAAAGCUCCGUCCCACCUU |
| SEQ ID No. 225 | GGUACUUUCAAAGCUCCGUCGC |
| SEQ ID No. 226 | GGUACUCCGCAUUCUUGGCGUA |
| SEQ ID No. 227 | GUAGCGUGAAUUUCAAACCCGC |
| SEQ ID No. 228 | GCGGGCGAACCCUUGAAACUCU |
| SEQ ID No. 229 | GCCGACACAAUUGUCGGCCCGU |
| SEQ ID No. 230 | GACGUUAACGCCCGCGGGUACU |
| SEQ ID No. 231 | GAAUCCGUCGGGCCCGGCGAUA |
| SEQ ID No. 232 | GCUCUUUACGUACUUCGGUAAU |
| SEQ ID No. 233 | CCCUAUUCAACAGAUCGUGGAU |
| SEQ ID No. 234 | GGGCCUCUCGUAACGGGUUGCA |
| SEQ ID No. 235 | CAUUAGCGGUAACUUGAGCCCG |
| SEQ ID No. 236 | CAUUAAUGUCGCAUAACGGCGA |
| SEQ ID No. 237 | GUUCAGGGAGCAUUUCCCGAAU |
| SEQ ID No. 238 | GGCGCCUAACGGACUCUUUAAA |
| SEQ ID No. 239 | GACGAAACCUUCCAGGACCCAA |
| SEQ ID No. 240 | CGUGGCUCCAAAUGACAGUAAC |
| SEQ ID No. 241 | CCCGAUUUCCAAUGACGGAAUU |
| SEQ ID No. 242 | GAUGUUAGGUUUCACGGUCCGU |
| SEQ ID No. 243 | GCAACCCGAAAGUUACGGCAGA |
| SEQ ID No. 244 | GAGUUUGCGUGACCCACGUAGG |
| SEQ ID No. 245 | GCCCGCUUGAACGCGUGACGAU |
| SEQ ID No. 246 | GUACUCUUCCGCGGCCAACGAU |
| SEQ ID No. 247 | CCUCUAUGUGGAACCUUUAAAG |
| SEQ ID No. 248 | GGGCCGGUCGAGUUUAACCAUU |
| SEQ ID No. 249 | UCCCGAUACCGGAAAGGCCAAG |
| SEQ ID No. 250 | ACGAUGGGUAACCGCACUUUAC |
| SEQ ID No. 251 | CAUUCGGUUAUCGUGAACAAGU |
| SEQ ID No. 252 | GCCCACGCAGUACUCGCGGAAU |
| SEQ ID No. 253 | GCUGGUAAUCCUUCGCGAAUUU |
| SEQ ID No. 254 | GGGCCAUCGACCCGCGUUUGCU |
| SEQ ID No. 255 | AGGGCUUUCGCCAAAUAGACGC |
| SEQ ID No. 256 | ACAACUCAUUCCAGCCCUAGGA |
| SEQ ID No. 257 | CAACCCUCGAGAAUACGGAACA |
| SEQ ID No. 258 | GACGUCCGCUCAUUCUCUAAUA |
| SEQ ID No. 259 | ACGCCUACCCGAAAUGUUGCCG |
| SEQ ID No. 260 | CACCCACUAUAAUGGCGGGUCG |
| SEQ ID No. 261 | GACGGCGUGCCUAAUUGGGUCG |
| SEQ ID No. 262 | AAUCUACAAUGACUUAGAAACG |
| SEQ ID No. 263 | GGGAACCUGAGCCCGUAACGCA |
| SEQ ID No. 264 | GUCGGUUGAUAACGCCACAAGC |
| SEQ ID No. 265 | CGAAAGGACACUCCUUCCAAUG |
| SEQ ID No. 266 | GUUUAAAGAUAUACGGAGCGAU |
| SEQ ID No. 267 | CUUAUUGGGUUGCGGUGCCAAA |
| SEQ ID No. 268 | GCGAGUUAGCUUCGAAACCAAU |
| SEQ ID No. 269 | CCGUUAACAAGUCGCGUAAGAG |
| SEQ ID No. 270 | CGCCUAAUAUACGUAGAGGCGG |
| SEQ ID No. 271 | GUUACUUCGAUACGCCCGACAA |
| SEQ ID No. 272 | CCUGGCCUCAGCUAGCCAAUUU |
| SEQ ID No. 273 | AAUAUGUGGCUAUCGCCAGCCG |
| SEQ ID No. 274 | AAAUGGGCGGGUCGAAUAUAAG |
| SEQ ID No. 275 | CAUCUAUUACCAACCGGAACAU |
| SEQ ID No. 276 | GACACAAGUGGUCUAUCACCAA |
| SEQ ID No. 277 | GUGUCCCGCGCUUGAGAACUAG |
| SEQ ID No. 278 | AGUAGCCCAGAGAAACAUAUCA |
| SEQ ID No. 279 | AGUAACUCCAUUUCCCGGACCC |
| SEQ ID No. 280 | GCCCACGAGUCGUAACCAUUAA |
| SEQ ID No. 281 | GCGCCUUUGGAGUAACCACGCU |
| SEQ ID No. 282 | UCCAACGAAACACGCUCCGACG |
| SEQ ID No. 283 | ACGCCGACUUACGGUUUAGUAA |
| SEQ ID No. 284 | GACUUGUUCAAUGACCGCCCGG |
| SEQ ID No. 285 | CACUGGUCGGGCCCGAAACUUG |
| SEQ ID No. 286 | GGAUUUACAUUCGCCCGUGUAG |
| SEQ ID No. 287 | AACGCCCACCUGAGUCUUAGCG |
| SEQ ID No. 288 | GCCAACCCGACGUAUUCAGAAG |
| SEQ ID No. 289 | GCCACGAGGUCCCGAAAUCGAC |
| SEQ ID No. 290 | GCUACUCGUUCCCGCCAACGGG |
| SEQ ID No. 291 | CCGAGCGUCGAAGUUCGUAAUA |
| SEQ ID No. 292 | GAGGCCAAGUUCCUGUAACCCA |
| SEQ ID No. 293 | GGUUCGAUAGGAGCGUAACAAA |
| SEQ ID No. 294 | GGGUCGAUAUCCAAAGCCCGCA |
| SEQ ID No. 295 | CCACGUUCUAAACCGAGACGCG |
| SEQ ID No. 296 | GGUCUAGUCACCUUUGAAUUCU |
| SEQ ID No. 297 | AUCUGACAGGUCCGACUCGCAA |
| SEQ ID No. 298 | GUAAUGGGCGUGGUACCCAUCC |
| SEQ ID No. 299 | CUAUCGUGAGCCCGCUAGACAG |
| SEQ ID No. 300 | GUCUGGGUUUAGUUCAUAACGG |
| SEQ ID No. 301 | GCUGUUAACUCCUAUAGCAAGU |
| SEQ ID No. 302 | AAGCGAUUCGUAACUAUAAACG |
| SEQ ID No. 303 | UCGACUCUACUUACCCGAACGA |
| SEQ ID No. 304 | GGGUUUCCUAGCCAAAUCGGAA |
| SEQ ID No. 305 | CGGGCCACACGCGCAUUUAAGC |
| SEQ ID No. 306 | UCUCUUAAGGGAACGGCCAAAG |
| SEQ ID No. 307 | GCAAGCCGUAGGCCCACAAACG |
| SEQ ID No. 308 | CUGGCGCUCGCCCUUACUUUAU |
| SEQ ID No. 309 | CGCUACGAAAUUGGUAAGUAUG |
| SEQ ID No. 310 | GGGUCAGACAUACGCAACGGCU |
| SEQ ID No. 311 | GAAGCGGGCCACAAACCUACUU |
| SEQ ID No. 312 | GAGGGCGAUCCUUACCCGUUUA |
| SEQ ID No. 313 | GGUCGAUUUACAUCCGCGUUGU |
| SEQ ID No. 314 | CUAACCACUUACGGGAGUAAAC |
| SEQ ID No. 315 | GAGUCCCAACUUCUAAUUUAGG |
| SEQ ID No. 316 | CCCAUAUACCUUUAUCCGGGUA |
| SEQ ID No. 317 | CCGUCACGACUGAUCAACCAGA |
| SEQ ID No. 318 | UGAUUAAGGCCCAAUGUUCAAA |
| SEQ ID No. 319 | CCUAACGAUGACGGAGGUCCAC |
| SEQ ID No. 320 | CCUCCAGCCGAUGAGUCGGUAA |
| SEQ ID No. 321 | GAACCGGUGCGCACGCUAAGUA |
| SEQ ID No. 322 | AUUGAUCCAAACCUUAACGGAC |
| SEQ ID No. 323 | GAUGGAUAAUCCGGGCUUAACU |
| SEQ ID No. 324 | GCCUUUGGACUAACCCAAGUAC |
| SEQ ID No. 325 | CCGUGGUACCCGCGCCAGCUAG |
| SEQ ID No. 326 | CGGUCGCGUAAUUUCCACACGU |
| SEQ ID No. 327 | CGCCUUCCGGGAAAGCCCUGCU |
| SEQ ID No. 328 | GCUUCCGUACGUUAUAUCAUUU |
| SEQ ID No. 329 | GCCGCUUUCGGUUAAGUCCAGG |
| SEQ ID No. 330 | CGGUCUCAAACCCGCGUUCUUA |

From within this set of sequences, additional examination can be performed to identify sequence pairs which would be suitable as primers. Criteria for individual sequences include 1) predicted Tm around 58-65° C. range under typical qPCR conditions (around 50 mM NaCl, 3 mM MgCl₂, and 0.8 mM dNTPs) (see Owczarzy et al., Biochemistry 47, 5336-5353 (2008); U.S. Patent Application 2009/0198453), 2) no significant self-hairpin potential, and 3) no significant self-dimer potential. In addition, pairwise examination must be performed to exclude those linker/primer pairs with significant heterodimer potential.

As used herein, the terms “nucleic acid” and “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base. There is no intended distinction in length between the terms “nucleic acid” and “oligonucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar, or phosphate backbone is modified as well as non-purine or non-pyrimidine nucleotide analogs oligonucleotides, which may comprise naturally occurring nucleosides or chemically modified nucleosides. In some embodiments, the compounds comprise modified sugar moieties, modified internucleoside linkages, or modified nucleobase moieties.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature. A primer is preferably a single-stranded DNA. The appropriate length of a primer depends on the intended use of the primer but typically ranges from 6 to 50 nucleotides, preferably from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein. Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product. The region of the primer which is sufficiently complementary to the template to hybridize is referred to herein as the hybridizing region.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barmy et al., U.S. Pat. No. 5,494,810). Exemplary “amplification reactions conditions” or “amplification conditions” typically comprise either two or three step cycles. Two step cycles have a high temperature denaturation step followed by a hybridization/elongation (or ligation) step. Three step cycles comprise a denaturation step followed by a hybridization step followed by a separate elongation or ligation step.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences which contain the target primer binding sites.

The term “primer dimer,” as used herein, refers to a template-independent non-specific amplification product, which is believed to result from primer extensions wherein another primer serves as a template. Although primer dimers frequently appear to be a concatamer of two primers, i.e., a dimer, concatamers of more than two primers also occur. The term “primer dimer” is used herein generically to encompass a template-independent non-specific amplification product.

The term “cleavage domain” or “cleaving domain,” as used herein, are synonymous and refer to a region located between the 5′ and 3′ end of a primer or other oligonucleotide that is recognized by a cleavage compound, for example a cleavage enzyme, that will cleave the primer or other oligonucleotide. For the purposes of this invention, the cleavage domain is designed such that the primer or other oligonucleotide is cleaved only when it is hybridized to a complementary nucleic acid sequence, but will not be cleaved when it is single-stranded. The cleavage domain or sequences flanking it may include a moiety that a) prevents or inhibits the extension or ligation of a primer or other oligonucleotide by a polymerase or a ligase, b) enhances discrimination to detect variant alleles, or c) suppresses undesired cleavage reactions. One or more such moieties may be included in the cleavage domain or the sequences flanking it.

An RNase H cleavage domain is a type of cleavage domain that contains one or more ribonucleic acid residue or an alternative analog which provides a substrate for an RNase H. An RNase H cleavage domain can be located anywhere within a primer or oligonucleotide. An RNase H2 cleavage domain may contain one RNA residue, a modified residue such as 2′-fluoronucleoside residue, a sequence of contiguously linked RNA residues, or RNA residues separated by DNA residues or other chemical groups. Other RNase H enzymes, such as RNase H1, can be utilized in the present invention. However, the cleavage domain of an RNase H1 enzyme requires at least 3 consecutive ribonucleotides.

Additional alternatives to an RNA residue that can be used in the present invention wherein cleavage is mediated by an RNase H enzyme include but are not limited to 2′-O-alkyl RNA nucleosides, preferably 2′-O-methyl RNA nucleosides, 2′-fluoronucleosides, locked nucleic acids (LNA), 2′-ENA residues (ethylene nucleic acids), 2′-alkyl nucleosides, 2′-aminonucleosides and 2′-thionucleosides. The RNase H cleavage domain may include one or more of these modified residues alone or in combination with RNA bases. DNA bases and abasic residues such as a C3 spacer may also be included to provide greater performance.

The term “blocking group,” as used herein, refers to a chemical moiety that is bound to the primer or other oligonucleotide such that an amplification or ligation reaction does not occur. For example, primer extension and/or RNA ligation does not occur. Once the blocking group is removed from the primer or other oligonucleotide, the oligonucleotide is capable of participating in the amplification or ligation assay for which it was designed. Thus, the “blocking group” can be any chemical moiety that inhibits recognition by a polymerase or RNA ligase. When referring to the blocking group on the primers of the current invention, the blocking group may be incorporated into the cleavage domain but is generally located on either the 5′- or 3′-side of the cleavage domain. The blocking group can be comprised of more than one chemical moiety. In the present invention the “blocking group” is typically removed after hybridization of the oligonucleotide to its target sequence.

For primer blocking groups, a number of blocking groups are known in the art that can be placed at or near the 3′ end of the oligonucleotide to prevent extension. A primer or other oligonucleotide may be modified at the 3′-terminal nucleotide to prevent or inhibit initiation of DNA synthesis by, for example, the addition of a 3′ deoxyribonucleotide residue (e.g., cordycepin), a 2′,3′-dideoxyribonucleotide residue, non-nucleotide linkages or alkane-diol modifications (U.S. Pat. No. 5,554,516). Alkane diol modifications which can be used to inhibit or block primer extension have also been described by Wilk et al., (1990, Nucleic Acids Res., 18 (8):2065), and by Arnold et al., (U.S. Pat. No. 6,031,091). Additional examples of suitable blocking groups include 3′ hydroxyl substitutions (e.g., 3′-phosphate, 3′-triphosphate or 3′-phosphate diesters with alcohols such as 3-hydroxypropyl), a 2′3′-cyclic phosphate, 2′ hydroxyl substitutions of a terminal RNA base (e.g., phosphate or sterically bulky groups such as triisopropyl silyl (TIPS) or tert-butyl dimethyl silyl (TBDMS)). 2′-alkyl silyl groups such as TIPS and TBDMS substituted at the 3′-end of an oligonucleotide are described by Laikhter et al., U.S. patent application Ser. No. 11/686,894 which is incorporated herein by reference. Bulky substituents can also be incorporated on the base of the 3′-terminal residue of the oligonucleotide to block primer extension.

Blocking groups to inhibit primer extension can also be located upstream, that is 5′, from the 3′-terminal residue. Sterically bulky substituents which interfere with binding by the polymerase can be incorporated onto the base, sugar or phosphate group of residues upstream from the 3′-terminus. Such substituents include bulky alkyl groups like t-butyl, triisopropyl and polyaromatic compounds including fluorophores and quenchers, and can be placed from one to about 10 residues from the 3′-terminus. Alternatively abasic residues such as a C3 spacer may be incorporated in these locations to block primer extension. In one such embodiment two adjacent C3 spacers have been employed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for detection of a target RNA in an RNA sample, the method comprising:
a) providing a reaction mixture comprising (i) an oligonucleotide primer having a cleavage domain positioned 5′ of a blocking group, (ii) an RNA sample that may or may not have the target RNA, (iii) a cleaving enzyme, (iv) a polymerase and (v) a dye that permits detection of an amplification product;
b) hybridizing the primer to the target RNA to form a double-stranded substrate;
c) cleaving the hybridized primer with said cleaving enzyme at a point within or adjacent to the cleavage domain to remove the blocking group from the primer;
d) extending the primer with the polymerase to form the amplification product; and
e) detecting the amplification product.

2. The method of claim 1 wherein the RNA sample is prepared by providing a reaction mixture comprising (i) isolated RNA from a sample, (ii) a 3′-linker capable of attachment to the 3′-end of the isolated RNA and wherein the 5′-end of the linker is adenylated, and (iii) an RNA ligase to form a 3′-linkered RNA sample.

3. The method of claim 2 wherein the RNA sample is further prepared by providing a reaction mixture comprising (i) the 3′-linkered RNA sample, (ii) a 5′-linker capable of attachment to the 5′-end of the 3′-linkered RNA sample, (iii) an RNA ligase, and (iv) ATP.

4. The method of claim 1 wherein the dye that permits detection of amplification product is a DNA binding dye.

5. The method of claim 4 wherein the DNA binding dye is a SYBR™ Green dye.

6. The method of claim 1 wherein the dye that permits detection of amplification product is a fluorophore that is attached to the oligonucleotide primer.

7. The method of claim 6 wherein a quencher is attached to the oligonucleotide primer on an opposing side of the cleavage domain from the fluorophore, wherein hybridization and cleavage of the primer increases fluorescence of the fluorophore.

8. The method of claim 2 wherein the oligonucleotide primer is capable of hybridizing to a portion of the 3′-linker and a portion of the target RNA adjacent to the 3′-linker.

9. The method of claim 3 wherein the oligonucleotide primer is capable of hybridizing to a portion of the 5′-linker and a portion of the target RNA adjacent to the 5′-linker.

10. The methods of claim 8 wherein a portion of the oligonucleotide primer containing the cleavable domain hybridizes to the target RNA.

11. The methods of claim 9 wherein a portion of the oligonucleotide primer containing the cleavable domain hybridizes to the target RNA.

12. The method of claim 1 wherein the RNA sample contains a control RNA, said control RNA comprising a sequence different from known miRNA sequences and is of equal length to an expected length of the target RNA.

13. The method of claim 3 wherein the sequence of at least one of the 3′ linker and the 5′ linker are selected from a group generated by:
a) generating a population of random 22mers from a uniform base distribution under the following conditions: (i) runs of length 4 or more of a single base are prohibited, (ii) consecutive doublet or triplet repeats of length 6 or more are prohibited, (iii) repeats of length 4 or more anywhere in the sequence are prohibited;
b) calculating the edit distance of each possible sequence against known miRNA sequences;
c) selecting a sequence with a minimum edit distance to a known miRNA sequence that is greater than 8.


SEQUENCE LISTING (330 sequences)

SEQ ID NO: 1 - DNA, 23 bp, Artificial
  Synthetic primer
ggctggagtgtagcagcacgnna

SEQ ID NO: 2 - DNA, 18 bp, Artificial
  Synthetic primer
ggctggagtgtagcagca

SEQ ID NO: 3 - DNA, 26 bp, Artificial
  Synthetic primer
attacgggatacggtggatcgccnnt

SEQ ID NO: 4 - DNA, 21 bp, Artificial
  Synthetic primer
attacgggatacggtggatcg

SEQ ID NO: 5 - DNA, 23 bp, Artificial
  Synthetic oligonucleotide
aatccaccgtatcccgtaatcan

SEQ ID NO: 6 - DNA, 23 bp, Artificial
  Synthetic oligonucelotide
ncaagtguucaaaggcuggagug

SEQ ID NO: 7 - DNA, 65 bp, Artificial
  Synthetic oligonucleotide
caagtgttcaaaggctggagtgtagcagcacgtaaatattggcgatccaccgtatcccgtaatca

SEQ ID NO: 8 - RNA, 22 nt, Artificial
  Synthetic oligonucleotide
uagcagcacguaaauauuggcg

SEQ ID NO: 9 - DNA, 21 bp, Artificial
  Synthetic oligonucleotide
tgattacgggatacggtggat

SEQ ID NO: 10 - RNA, 22 nt, Artificial
  Synthetic oligonucleotide
gguuucggagaacccgugggcu

SEQ ID NO: 11 - RNA, 22 nt, Artificial
  Synthetic oligonucleotide
gaguaaauucgcucaacuacgu

SEQ ID NO: 12 - RNA, 22 nt, Artificial
  Synthetic oligonucleotide
acuuuccgguaggauuaaccaa

SEQ ID NO: 13 - RNA, 22 nt, Artificial
  Synthetic oligonucleotide
ccgaauuuauccucggcgauug

SEQ ID NO: 14 - RNA, 22 nt, Artificial
  Synthetic oligonucleotide
cuuaucuacaaguacgcgauaa

SEQ ID NO: 15 - RNA, 22 nt, Artificial
  Synthetic oligonucleotide
gcugacguauaaaugcccucga

SEQ ID NO: 16 - RNA, 22 nt, Artificial
  Synthetic oligonucleotide
cagcaagaggcgacccaaaggu

SEQ ID NO: 17 - RNA, 22 nt, Artificial
  Synthetic oligonucleotide
gaacguuggauacgcccgugua

SEQ ID NO: 18 - RNA, 22 nt, Artificial
  Synthetic oligonucleotide
ccgccuccauuucaaccuagaa

SEQ ID NO: 19 - RNA, 22 nt, Artificial
  Synthetic oligonucleotide
cccgcgccuucggaugcauggg

SEQ ID NO: 20 - RNA, 22 nt, Artificial
  Synthetic oligonucleotide
gacucuaagugagccagggucg

SEQ ID NO: 21 - RNA, 22 nt, Artificial
  Synthetic oligonucleotide
auaaccauaucgccucccuggg

SEQ ID NO: 22 - RNA, 22 nt, Artificial
  Synthetic oligonucleotide
gcccgagcucuaccgcgucaaa

SEQ ID NO: 23 - RNA, 22 nt, Artificial
  Synthetic oligonucleotide
gccguguuacuaaacuccuugg

SEQ ID NO: 24 - RNA, 22 nt, Artificial
  Synthetic oligonucleotide
guauggccaaauuagccucgaa

SEQ ID NO: 25 - RNA, 22 nt, Artificial
  Synthetic oligonucleotide
caauuaaggagucauucacgcc

SEQ ID NO: 26 - RNA, 22 nt, Artificial
  Synthetic oligonucleotide
ccuauauuccgaucgagcacga

SEQ ID NO: 27 - RNA, 22 nt, Artificial
  Synthetic oligonucleotide
ggcagcuuaguuaaucuauaaa

SEQ ID NO: 28 - RNA, 22 nt, Artificial
  Synthetic oligonucleotide
gacuuugccucaagggacgauu

SEQ ID NO: 29 - RNA, 22 nt, Artificial
  Synthetic oligonucleotide
gcgcuacagacaugcggcccaa

SEQ ID NO: 30 - RNA, 22 nt, Artificial
  Synthetic oligonucleotide
gccguccgggauucuuaaauua

SEQ ID NO: 31 - RNA, 22 nt, Artificial
  Synthetic oligonucleotide
ccaucucuacgacacguaggaa

SEQ ID NO: 32 - RNA, 22 nt, Artificial
  Synthetic oligonucleotide
ggcgcauaagaauccaacgauu

SEQ ID NO: 33 - RNA, 22 nt, Artificial
  Synthetic oligonucleotide
guguuuaacuuuggcuacgccc

SEQ ID NO: 34 - RNA, 22 nt, Artificial
  Synthetic oligonucleotide
ggcgcgacccuuuaaacuaucc

SEQ ID NO: 35 - RNA, 22 nt, Artificial
  Synthetic oligonucleotide
ccaagggccgaaucauuccgua

SEQ ID NO: 36 - RNA, 22 nt, Artificial
  Synthetic oligonucleotide
aauaucgcguaacccgcucuag

SEQ ID NO: 37 - RNA, 22 nt, Artificial
  Synthetic oligonucleotide
ugguuaagggcuucccgaaagc

SEQ ID NO: 38 - RNA, 22 nt, Artificial
  Synthetic oligonucleotide
accuuucucguacacgcgcaua

SEQ ID NO: 39 - RNA, 22 nt, Artificial
  Synthetic oligonucleotide
ggcguguggaauccgccaaccg

SEQ ID NO: 40 - RNA, 22 nt, Artificial
  Synthetic oligonucleotide
gagaaaucuuacacaggccauc

SEQ ID NO: 41 - RNA, 22 nt, Artificial
  Synthetic oligonucleotide
ggacccuuccgaaacuuuauaa

SEQ ID NO: 42 - RNA, 22 nt, Artificial
  Synthetic oligonucleotide
gggaguaucgcccauuacuuua

SEQ ID NO: 43 - RNA, 22 nt, Artificial
  Synthetic oligonucleotide
gagaacagggccuaugauaucc

SEQ ID NO: 44 - RNA, 22 nt, Artificial
  Synthetic oligonucleotide
auucuaccacaaugucgcguua

SEQ ID NO: 45 - RNA, 22 nt, Artificial
  Synthetic oligonucleotide
gcucgaacggccaagacauaug

SEQ ID NO: 46 - RNA, 22 nt, Artificial
  Synthetic oligonucleotide
acccaagcuaaccgauacgccc

SEQ ID NO: 47 - RNA, 22 nt, Artificial
  Synthetic oligonucleotide
ccgacguuuagcccaaccuccc

SEQ ID NO: 48 - RNA, 22 nt, Artificial
  Synthetic oligonucleotide
accuuagguuuaccgggccgac

SEQ ID NO: 49 - RNA, 22 nt, Artificial
  Synthetic oligonucleotide
gccguuccaggacgcacuaaag

SEQ ID NO: 50 - RNA, 22 nt, Artificial
  Synthetic oligonucleotide
cccuccggguuugucaauaagg

SEQ ID NO: 51 - RNA, 22 nt, Artificial
  Synthetic oligonucleotide
gauacguuccgaacaauaaccg

SEQ ID NO: 52 - RNA, 22 nt, Artificial
  Synthetic oligonucleotide
gcaauacucaucuuaaauccua

SEQ ID NO: 53 - RNA, 22 nt, Artificial
  Synthetic oligonucleotide
ccgauucugcucuuugaaaggg

SEQ ID NO: 54 - RNA, 22 nt, Artificial
  Synthetic oligonucleotide
ccgauccggcgagcgucaacau

SEQ ID NO: 55 - RNA, 22 nt, Artificial
  Synthetic oligonucleotide
gcuuuagggcuacguauagacc

SEQ ID NO: 56 - RNA, 22 nt, Artificial
  Synthetic oligonucleotide
aagggcuccuaagcaacccguc

SEQ ID NO: 57 - RNA, 22 nt, Artificial
  Synthetic oligonucleotide
cugccaguggguacggcaaaug

SEQ ID NO: 58 - RNA, 22 nt, Artificial
  Synthetic oligonucleotide
gggaagggugauuccuuaaaug

SEQ ID NO: 59 - RNA, 22 nt, Artificial
  Synthetic oligonucleotide
gaccagacgcccucgcgucaau

SEQ ID NO: 60 - RNA, 22 nt, Artificial
  Synthetic oligonucleotide
gaagcaaccucaauuuacacug

SEQ ID NO: 61 - RNA, 22 nt, Artificial
  Synthetic oligonucleotide
cccuaagcguccuuccgaaaua

SEQ ID NO: 62 - RNA, 22 nt, Artificial
  Synthetic oligonucleotide
auugcgggcugccgguccaaag

SEQ ID NO: 63 - RNA, 22 nt, Artificial
  Synthetic oligonucleotide
acauacacgguccuuaugcgua

SEQ ID NO: 64 - RNA, 22 nt, Artificial
  Synthetic oligonucleotide
aacaaacgggauuugauauacg

SEQ ID NO: 65 - RNA, 22 nt, Artificial
  Synthetic oligonucleotide
gguuucgcuuacuauaaccaau

SEQ ID NO: 66 - RNA, 22 nt, Artificial
  Synthetic oligonucleotide
gcguuugcacgcuuauaaauua

SEQ ID NO: 67 - RNA, 22 nt, Artificial
  Synthetic oligonucleotide
guuccaaauauuuaggccuaau

SEQ ID NO: 68 - RNA, 22 nt, Artificial
  Synthetic oligonucleotide
acggcgacguaaacgaacccuc

SEQ ID NO: 69 - RNA, 22 nt, Artificial
  Synthetic oligonucleotide
guagacgaaaucaagccaccgc

SEQ ID NO: 70 - RNA, 22 nt, Artificial
  Synthetic oligonucleotide
ccuuugcuguaacuuaagcgcc

SEQ ID NO: 71 - RNA, 22 nt, Artificial
  Synthetic oligonucleotide
ggcccuauggagugugccaauu

SEQ ID NO: 72 - RNA, 22 nt, Artificial
  Synthetic oligonucleotide
auaacgcuugcgccgaucaacu

SEQ ID NO: 73 - RNA, 22 nt, Artificial
  Synthetic oligonucleotide
gucgcuacgguaacccagucac

SEQ ID NO: 74 - RNA, 22 nt, Artificial
  Synthetic oligonucleotide
guaugggccucgaaagggaucg

SEQ ID NO: 75 - RNA, 22 nt, Artificial
  Synthetic oligonucleotide
acccgucgacuuacggacaaau

SEQ ID NO: 76 - RNA, 22 nt, Artificial
  Synthetic oligonucleotide
acuacgcuccggauauuagacc

SEQ ID NO: 77 - RNA, 22 nt, Artificial
  Synthetic oligonucleotide
caccgucaagguuauaaacaaa

SEQ ID NO: 78 - RNA, 22 nt, Artificial
  Synthetic oligonucleotide
ggcauacaacgacccuagugau

SEQ ID NO: 79 - RNA, 22 nt, Artificial
  Synthetic oligonucleotide
gggaugcggucuccuaaacgcg

SEQ ID NO: 80 - RNA, 22 nt, Artificial
  Synthetic oligonucleotide
gcuuauaccguugaauaaacgu

SEQ ID NO: 81 - RNA, 22 nt, Artificial
  Synthetic oligonucleotide
guuacgauugguccauaacaga

SEQ ID NO: 82 - RNA, 22 nt, Artificial
  Synthetic oligonucleotide
ccgcgcaauggguggauacaag

SEQ ID NO: 83 - RNA, 22 nt, Artificial
  Synthetic oligonucleotide
gcggaacaauuaucuuagagcc

SEQ ID NO: 84 - RNA, 22 nt, Artificial
  Synthetic oligonucleotide
cccgccaauaaccucgaaggaa

SEQ ID NO: 85 - RNA, 22 nt, Artificial
  Synthetic oligonucleotide
cucgcguuauaccagaaggagu

SEQ ID NO: 86 - RNA, 22 nt, Artificial
  Synthetic oligonucleotide
aagaauaaacggcccaucgcua

SEQ ID NO: 87 - RNA, 22 nt, Artificial
  Synthetic oligonucleotide
cgcaauuagaccaguuaacggg

SEQ ID NO: 88 - RNA, 22 nt, Artificial
  Synthetic oligonucleotide
cguauacagcggccgcucaacg

SEQ ID NO: 89 - RNA, 22 nt, Artificial
  Synthetic oligonucleotide
gcccaacauccggucagguaaa

SEQ ID NO: 90 - RNA, 22 nt, Artificial
  Synthetic oligonucleotide
gguguccggauaauucaaacua

SEQ ID NO: 91 - RNA, 22 nt, Artificial
  Synthetic oligonucleotide
cgccggcgaagcuuaacagaaa

SEQ ID NO: 92 - RNA, 22 nt, Artificial
  Synthetic oligonucleotide
ggcguaauacaaucuccaugcc

SEQ ID NO: 93 - RNA, 22 nt, Artificial
  Synthetic oligonucleotide
gauauaaucagaucccgggugg

SEQ ID NO: 94 - RNA, 22 nt, Artificial
  Synthetic oligonucleotide
ccacuauccaaucaacguuagg

SEQ ID NO: 95 - RNA, 22 nt, Artificial
  Synthetic oligonucleotide
ccgacucuaagaggcacaaggu

SEQ ID NO: 96 - RNA, 22 nt, Artificial
  Synthetic oligonucleotide
ggccuuuagugaaccgcgcauc

SEQ ID NO: 97 - RNA, 22 nt, Artificial
  Synthetic oligonucleotide
caaccaauaucauaagaucguc

SEQ ID NO: 98 - RNA, 22 nt, Artificial
  Synthetic oligonucleotide
gcccggaaauaagaccaagugg

SEQ ID NO: 99 - RNA, 22 nt, Artificial
  Synthetic oligonucleotide
cgucgaacuccaauuaaagauc

SEQ ID NO: 100 - RNA, 22 nt, Artificial
  Synthetic oligonucleotide
gccuuuaggcuacccucaauua

SEQ ID NO: 101 - RNA, 22 nt, Artificial
  Synthetic oligonucleotide
guacacuacgggcccacgacca

SEQ ID NO: 102 - RNA, 22 nt, Artificial
  Synthetic oligonucleotide
gucacaauuuaaaccagauucg

SEQ ID NO: 103 - RNA, 22 nt, Artificial
  Synthetic oligonucleotide
gugucguuacucaccuaauuug

SEQ ID NO: 104 - RNA, 22 nt, Artificial
  Synthetic oligonucleotide
gacuugccuuaacccuauacug

SEQ ID NO: 105 - RNA, 22 nt, Artificial
  Synthetic oligonucleotide
aauaacauagggccauucuuaa

SEQ ID NO: 106 - RNA, 22 nt, Artificial
  Synthetic oligonucleotide
gguuacauaccgcccuagcguu

SEQ ID NO: 107 - RNA, 22 nt, Artificial
  Synthetic oligonucleotide
gguguuuacggcuaaacguaag

SEQ ID NO: 108 - RNA, 22 nt, Artificial
  Synthetic oligonucleotide
gagcgaguucuuucgacgggcc

SEQ ID NO: 109 - RNA, 22 nt, Artificial
  Synthetic oligonucleotide
gaaacggaacaauccaccuucu

SEQ ID NO: 110 - RNA, 22 nt, Artificial
  Synthetic oligonucleotide
uuuccgguagugacccuacgua

SEQ ID NO: 111 - RNA, 22 nt, Artificial
  Synthetic oligonucleotide
cguacggacgcucagaaucaac

SEQ ID NO: 112 - RNA, 22 nt, Artificial
  Synthetic oligonucleotide
cgccaaccucggcuaaucguaa

SEQ ID NO: 113 - RNA, 22 nt, Artificial
  Synthetic oligonucleotide
ggaagagguguucccgcggcac

SEQ ID NO: 114 - RNA, 22 nt, Artificial
  Synthetic oligonucleotide
ggcccuucauacucgugaaacg

SEQ ID NO: 115 - RNA, 22 nt, Artificial
  Synthetic oligonucleotide
ugauccaagcgcuuaugcaaua

SEQ ID NO: 116 - RNA, 22 nt, Artificial
  Synthetic oligonucleotide
gaaacugauuagauacccgccc

SEQ ID NO: 117 - RNA, 22 nt, Artificial
  Synthetic oligonucleotide
ccaaggcgacguccauauccgg

SEQ ID NO: 118 - RNA, 22 nt, Artificial
  Synthetic oligonucleotide
ccgcgcuuucgagggcccguau

SEQ ID NO: 119 - RNA, 22 nt, Artificial
  Synthetic oligonucleotide
gaacaccuaacggcgugguagg

SEQ ID NO: 120 - RNA, 22 nt, Artificial
  Synthetic oligonucleotide
aaacggagaaccacuagcuaag

SEQ ID NO: 121 - RNA, 22 nt, Artificial
  Synthetic oligonucleotide
uguuauaucaagcccuagggaa

SEQ ID NO: 122 - RNA, 22 nt, Artificial
  Synthetic oligonucleotide
guucagcgcgaacgcaauccga

SEQ ID NO: 123 - RNA, 22 nt, Artificial
  Synthetic oligonucleotide
gacucaucgcaauaggcgggaa

SEQ ID NO: 124 - RNA, 22 nt, Artificial
  Synthetic oligonucleotide
ggguggauugggacccugcccg

SEQ ID NO: 125 - RNA, 22 nt, Artificial
  Synthetic oligonucleotide
ccgucgagaggaugguucuuua

SEQ ID NO: 126 - RNA, 22 nt, Artificial
  Synthetic oligonucleotide
auuuggcccuaacuauauccgg

SEQ ID NO: 127 - RNA, 22 nt, Artificial
  Synthetic oligonucleotide
cccuagguauccggcgcaagua

SEQ ID NO: 128 - RNA, 22 nt, Artificial
  Synthetic oligonucleotide
ccgguccacaaucccagagaua

SEQ ID NO: 129 - RNA, 22 nt, Artificial
  Synthetic oligonucleotide
gguuuaacauacgcgaagcuaa

SEQ ID NO: 130 - RNA, 22 nt, Artificial
  Synthetic oligonucleotide
gaaaucccauacuauucgcccg

SEQ ID NO: 131 - RNA, 22 nt, Artificial
  Synthetic oligonucleotide
cucgcgaccaaauccguuuaaa

SEQ ID NO: 132 - RNA, 22 nt, Artificial
  Synthetic oligonucleotide
cccaugcuuacgauaggcgaaa

SEQ ID NO: 133 - RNA, 22 nt, Artificial
  Synthetic oligonucleotide
ggcuugcccacagauuuaaagg

SEQ ID NO: 134 - RNA, 22 nt, Artificial
  Synthetic oligonucleotide
gccggcgaaccuaucaacguua

SEQ ID NO: 135 - RNA, 22 nt, Artificial
  Synthetic oligonucleotide
gacgcgaauaacuucaucucua

SEQ ID NO: 136 - RNA, 22 nt, Artificial
  Synthetic oligonucleotide
agacgccucaacuguuacccga

SEQ ID NO: 137 - RNA, 22 nt, Artificial
  Synthetic oligonucleotide
gguccuuacguagguugggaaa

SEQ ID NO: 138 - RNA, 22 nt, Artificial
  Synthetic oligonucleotide
ggcaacuccgcagcuuauuuac

SEQ ID NO: 139 - RNA, 22 nt, Artificial
  Synthetic oligonucleotide
ggcccaaaccggaaucgaggga

SEQ ID NO: 140 - RNA, 22 nt, Artificial
  Synthetic oligonucleotide
ggcccgcgaacguaccuaaaua

SEQ ID NO: 141 - RNA, 22 nt, Artificial
  Synthetic oligonucleotide
cuacgagcauuuagcccgucac

SEQ ID NO: 142 - RNA, 22 nt, Artificial
  Synthetic oligonucleotide
gaguucgucuaagaugagaccc

SEQ ID NO: 143 - RNA, 22 nt, Artificial
  Synthetic oligonucleotide
cggauuggguuccaaccacaaa

SEQ ID NO: 144 - RNA, 22 nt, Artificial
  Synthetic oligonucleotide
gacacgucgcgcaacuaaagag

SEQ ID NO: 145 - RNA, 22 nt, Artificial
  Synthetic oligonucleotide
gcucgcguagacacuauccgau

SEQ ID NO: 146 - RNA, 22 nt, Artificial
  Synthetic oligonucleotide
cugccggccuauucaaguaaag

SEQ ID NO: 147 - RNA, 22 nt, Artificial
  Synthetic oligonucleotide
guuugaaaccggacagguuccu

SEQ ID NO: 148 - RNA, 22 nt, Artificial
  Synthetic oligonucleotide
aucccgacggcgaaagcuaucg

SEQ ID NO: 149 - RNA, 22 nt, Artificial
  Synthetic oligonucleotide
caagacgucacgaaccgagcaa

SEQ ID NO: 150 - RNA, 22 nt, Artificial
  Synthetic oligonucleotide
ggcaccgucggaucacgaauau

SEQ ID NO: 151 - RNA, 22 nt, Artificial
  Synthetic oligonucleotide
gaucaguuucacccgaaccaaa

SEQ ID NO: 152 - RNA, 22 nt, Artificial
  Synthetic oligonucleotide
gacgcucgguaaguuaaccaag

SEQ ID NO: 153 - RNA, 22 nt, Artificial
  Synthetic oligonucleotide
uuaaucggguucccgcguggcu

SEQ ID NO: 154 - RNA, 22 nt, Artificial
  Synthetic oligonucleotide
ccuacgaguacuuaacccgucc

SEQ ID NO: 155 - RNA, 22 nt, Artificial
  Synthetic oligonucleotide
cauacgcgaucugcccaaaccu

SEQ ID NO: 156 - RNA, 22 nt, Artificial
  Synthetic oligonucleotide
aaacgguugagcuacgcuuaua

SEQ ID NO: 157 - RNA, 22 nt, Artificial
  Synthetic oligonucleotide
caauucccgccagcgcgugggu

SEQ ID NO: 158 - RNA, 22 nt, Artificial
  Synthetic oligonucleotide
guggcucauaccggucaauccc

SEQ ID NO: 159 - RNA, 22 nt, Artificial
  Synthetic oligonucleotide
cacgauaguuaagcaacccgcc

SEQ ID NO: 160 - RNA, 22 nt, Artificial
  Synthetic oligonucleotide
ggaccguuaaacggugugcgcg

SEQ ID NO: 161 - RNA, 22 nt, Artificial
  Synthetic oligonucleotide
ccggagcauuggccacgagugc

SEQ ID NO: 162 - RNA, 22 nt, Artificial
  Synthetic oligonucleotide
cgaggcucccucaacgguaaag

SEQ ID NO: 163 - RNA, 22 nt, Artificial
  Synthetic oligonucleotide
ccugguguauguuucgacaccg

SEQ ID NO: 164 - RNA, 22 nt, Artificial
  Synthetic oligonucleotide
ccgcccggaauguacaauaaac

SEQ ID NO: 165 - RNA, 22 nt, Artificial
  Synthetic oligonucleotide
ggcgauaaacguggcccuuagg

SEQ ID NO: 166 - RNA, 22 nt, Artificial
  Synthetic oligonucleotide
gcgggaaccuauagacucaggg

SEQ ID NO: 167 - RNA, 22 nt, Artificial
  Synthetic oligonucleotide
gccuuaauggcguucgcgccgu

SEQ ID NO: 168 - RNA, 22 nt, Artificial
  Synthetic oligonucleotide
gacuuacagacgcgccgagaaa

SEQ ID NO: 169 - RNA, 22 nt, Artificial
  Synthetic oligonucleotide
ccgcaagcuuugggaauuauaa

SEQ ID NO: 170 - RNA, 22 nt, Artificial
  Synthetic oligonucleotide
gacgcugcccucuacaucgcga

SEQ ID NO: 171 - RNA, 22 nt, Artificial
  Synthetic oligonucleotide
ccgcgccaaaccaucgaaguug

SEQ ID NO: 172 - RNA, 22 nt, Artificial
  Synthetic oligonucleotide
gaaucccgcccacuaucgcgac

SEQ ID NO: 173 - RNA, 22 nt, Artificial
  Synthetic oligonucleotide
ggucaaccggauuacuauggua

SEQ ID NO: 174 - RNA, 22 nt, Artificial
  Synthetic oligonucleotide
gggaugagaaguucgcacccaa

SEQ ID NO: 175 - RNA, 22 nt, Artificial
  Synthetic oligonucleotide
gauauugccagcuccgguuaac

SEQ ID NO: 176 - RNA, 22 nt, Artificial
  Synthetic oligonucleotide
cgcauuaucuagaaccgucaga

SEQ ID NO: 177 - RNA, 22 nt, Artificial
  Synthetic oligonucleotide
gucacagaaucgaccuuaaagu

SEQ ID NO: 178 - RNA, 22 nt, Artificial
  Synthetic oligonucleotide
cacgguaaucagccgacgcaau

SEQ ID NO: 179 - RNA, 22 nt, Artificial
  Synthetic oligonucleotide
ccgcccuaaaucugggccaaug

SEQ ID NO: 180 - RNA, 22 nt, Artificial
  Synthetic oligonucleotide
agguaauagcacuaacccgccc

SEQ ID NO: 181 - RNA, 22 nt, Artificial
  Synthetic oligonucleotide
ggcuccuuauccguagcggaaa

SEQ ID NO: 182 - RNA, 22 nt, Artificial
  Synthetic oligonucleotide
acuuacucaauagccuuuagag

SEQ ID NO: 183 - RNA, 22 nt, Artificial
  Synthetic oligonucleotide
guauaauugcaaccgccucgac

SEQ ID NO: 184 - RNA, 22 nt, Artificial
  Synthetic oligonucleotide
ccuacccaaaggcggacgcgac

SEQ ID NO: 185 - RNA, 22 nt, Artificial
  Synthetic oligonucleotide
accgucggaccaugcguaauac

SEQ ID NO: 186 - RNA, 22 nt, Artificial
  Synthetic oligonucleotide
cgcgggaaaguaccggacuaaa

SEQ ID NO: 187 - RNA, 22 nt, Artificial
  Synthetic oligonucleotide
cccucgcucuacguuaaggcug

SEQ ID NO: 188 - RNA, 22 nt, Artificial
  Synthetic oligonucleotide
auuaacccagacaucuucgagc

SEQ ID NO: 189 - RNA, 22 nt, Artificial
  Synthetic oligonucleotide
cugccguuccggguugagcaaa

SEQ ID NO: 190 - RNA, 22 nt, Artificial
  Synthetic oligonucleotide
caaauccgguacccuuaaacua

SEQ ID NO: 191 - RNA, 22 nt, Artificial
  Synthetic oligonucleotide
cggucugcuuucggaauaaagc

SEQ ID NO: 192 - RNA, 22 nt, Artificial
  Synthetic oligonucleotide
gaagggaauaucucggacaacc

SEQ ID NO: 193 - RNA, 22 nt, Artificial
  Synthetic oligonucleotide
aaucggacagcgcaaacggccc

SEQ ID NO: 194 - RNA, 22 nt, Artificial
  Synthetic oligonucleotide
gggauacccaagaaauaguccc

SEQ ID NO: 195 - RNA, 22 nt, Artificial
  Synthetic oligonucleotide
gguaccacgaaaucaaggcaca

SEQ ID NO: 196 - RNA, 22 nt, Artificial
  Synthetic oligonucleotide
cucggcccacgcaguuuaaggu

SEQ ID NO: 197 - RNA, 22 nt, Artificial
  Synthetic oligonucleotide
gaccgaaacagucguguugacg

SEQ ID NO: 198 - RNA, 22 nt, Artificial
  Synthetic oligonucleotide
gguagggcguccgcccuaacuc

SEQ ID NO: 199 - RNA, 22 nt, Artificial
  Synthetic oligonucleotide
gcaguaaagccguauaaggcca

SEQ ID NO: 200 - RNA, 22 nt, Artificial
  Synthetic oligonucleotide
gucaauuucucuuacccgaaca

SEQ ID NO: 201 - RNA, 22 nt, Artificial
  Synthetic oligonucleotide
gcgaaauggcauaccacuccca

SEQ ID NO: 202 - RNA, 22 nt, Artificial
  Synthetic oligonucleotide
ccauagcuuugcggccaacuag

SEQ ID NO: 203 - RNA, 22 nt, Artificial
  Synthetic oligonucleotide
ccauagcuuugcggccaacuag

SEQ ID NO: 204 - RNA, 22 nt, Artificial
  Synthetic oligonucleotide
acgaacggcuaaccuaccauaa

SEQ ID NO: 205 - RNA, 22 nt, Artificial
  Synthetic oligonucleotide
gcuccggaucaacguggcgaag

SEQ ID NO: 206 - RNA, 22 nt, Artificial
  Synthetic oligonucleotide
aaacgaaucccucauuuauacu

SEQ ID NO: 207 - RNA, 22 nt, Artificial
  Synthetic oligonucleotide
cgcuuaguaaugaccucacgcc

SEQ ID NO: 208 - RNA, 22 nt, Artificial
  Synthetic oligonucleotide
gcccauguuaagcguaaauaga

SEQ ID NO: 209 - RNA, 22 nt, Artificial
  Synthetic oligonucleotide
gauauugccgucgccuaggucc

SEQ ID NO: 210 - RNA, 22 nt, Artificial
  Synthetic oligonucleotide
augaagucccuaauaccaaccc

SEQ ID NO: 211 - RNA, 22 nt, Artificial
  Synthetic oligonucleotide
ccugauguuuagcgcucagguu

SEQ ID NO: 212 - RNA, 22 nt, Artificial
  Synthetic oligonucleotide
gggauacacguuccaaccggaa

SEQ ID NO: 213 - RNA, 22 nt, Artificial
  Synthetic oligonucleotide
ggcacuuuacguccuuggccga

SEQ ID NO: 214 - RNA, 22 nt, Artificial
  Synthetic oligonucleotide
gggaacguccggcaacuuaaau

SEQ ID NO: 215 - RNA, 22 nt, Artificial
  Synthetic oligonucleotide
cccgcucccuuaacugcgaauu

SEQ ID NO: 216 - RNA, 22 nt, Artificial
  Synthetic oligonucleotide
cgguccgcggcaugccacgaaa

SEQ ID NO: 217 - RNA, 22 nt, Artificial
  Synthetic oligonucleotide
ggugaaggccaaacccugcaau

SEQ ID NO: 218 - RNA, 22 nt, Artificial
  Synthetic oligonucleotide
gccauauggcucccggaaaucg

SEQ ID NO: 219 - RNA, 22 nt, Artificial
  Synthetic oligonucleotide
cgugcccagauaauggauuuag

SEQ ID NO: 220 - RNA, 22 nt, Artificial
  Synthetic oligonucleotide
gguucaaacgcguaccuggcgg

SEQ ID NO: 221 - RNA, 22 nt, Artificial
  Synthetic oligonucleotide
gacaaauucccgaggguucgcg

SEQ ID NO: 222 - RNA, 22 nt, Artificial
  Synthetic oligonucleotide
acaacuacgcggaccgaagcaa

SEQ ID NO: 223 - RNA, 22 nt, Artificial
  Synthetic oligonucleotide
ccgccauuaaggcuacgcaaau

SEQ ID NO: 224 - RNA, 22 nt, Artificial
  Synthetic oligonucleotide
ggauaaagcuccgucccaccuu

SEQ ID NO: 225 - RNA, 22 nt, Artificial
  Synthetic oligonucleotide
gguacuuucaaagcuccgucgc

SEQ ID NO: 226 - RNA, 22 nt, Artificial
  Synthetic oligonucleotide
gguacuccgcauucuuggcgua

SEQ ID NO: 227 - RNA, 22 nt, Artificial
  Synthetic oligonucleotide
guagcgugaauuucaaacccgc

SEQ ID NO: 228 - RNA, 22 nt, Artificial
  Synthetic oligonucleotide
gcgggcgaacccuugaaacucu

SEQ ID NO: 229 - RNA, 22 nt, Artificial
  Synthetic oligonucleotide
gccgacacaauugucggcccgu

SEQ ID NO: 230 - RNA, 22 nt, Artificial
  Synthetic oligonucleotide
gacguuaacgcccgcggguacu

SEQ ID NO: 231 - RNA, 22 nt, Artificial
  Synthetic oligonucleotide
gaauccgucgggcccggcgaua

SEQ ID NO: 232 - RNA, 22 nt, Artificial
  Synthetic oligonucleotide
gcucuuuacguacuucgguaau

SEQ ID NO: 233 - RNA, 22 nt, Artificial
  Synthetic oligonucleotide
cccuauucaacagaucguggau

SEQ ID NO: 234 - RNA, 22 nt, Artificial
  Synthetic oligonucleotide
gggccucucguaacggguugca

SEQ ID NO: 235 - RNA, 22 nt, Artificial
  Synthetic oligonucleotide
cauuagcgguaacuugagcccg

SEQ ID NO: 236 - RNA, 22 nt, Artificial
  Synthetic oligonucleotide
cauuaaugucgcauaacggcga

SEQ ID NO: 237 - RNA, 22 nt, Artificial
  Synthetic oligonucleotide
guucagggagcauuucccgaau

SEQ ID NO: 238 - RNA, 22 nt, Artificial
  Synthetic oligonucleotide
ggcgccuaacggacucuuuaaa

SEQ ID NO: 239 - RNA, 22 nt, Artificial
  Synthetic oligonucleotide
gacgaaaccuuccaggacccaa

SEQ ID NO: 240 - RNA, 22 nt, Artificial
  Synthetic oligonucleotide
cguggcuccaaaugacaguaac

SEQ ID NO: 241 - RNA, 22 nt, Artificial
  Synthetic oligonucleotide
cccgauuuccaaugacggaauu

SEQ ID NO: 242 - RNA, 22 nt, Artificial
  Synthetic oligonucleotide
gauguuagguuucacgguccgu

SEQ ID NO: 243 - RNA, 22 nt, Artificial
  Synthetic oligonucleotide
gcaacccgaaaguuacggcaga

SEQ ID NO: 244 - RNA, 22 nt, Artificial
  Synthetic oligonucleotide
gaguuugcgugacccacguagg

SEQ ID NO: 245 - RNA, 22 nt, Artificial
  Synthetic oligonucleotide
gcccgcuugaacgcgugacgau

SEQ ID NO: 246 - RNA, 22 nt, Artificial
  Synthetic oligonucleotide
guacucuuccgcggccaacgau

SEQ ID NO: 247 - RNA, 22 nt, Artificial
  Synthetic oligonucleotide
ccucuauguggaaccuuuaaag

SEQ ID NO: 248 - RNA, 22 nt, Artificial
  Synthetic oligonucleotide
gggccggucgaguuuaaccauu

SEQ ID NO: 249 - RNA, 22 nt, Artificial
  Synthetic oligonucleotide
ucccgauaccggaaaggccaag

SEQ ID NO: 250 - RNA, 22 nt, Artificial
  Synthetic oligonucleotide
acgauggguaaccgcacuuuac

SEQ ID NO: 251 - RNA, 22 nt, Artificial
  Synthetic oligonucleotide
cauucgguuaucgugaacaagu

SEQ ID NO: 252 - RNA, 22 nt, Artificial
  Synthetic oligonucleotide
gcccacgcaguacucgcggaau

SEQ ID NO: 253 - RNA, 22 nt, Artificial
  Synthetic oligonucleotide
gcugguaauccuucgcgaauuu

SEQ ID NO: 254 - RNA, 22 nt, Artificial
  Synthetic oligonucleotide
gggccaucgacccgcguuugcu

SEQ ID NO: 255 - RNA, 22 nt, Artificial
  Synthetic oligonucleotide
agggcuuucgccaaauagacgc

SEQ ID NO: 256 - RNA, 22 nt, Artificial
  Synthetic oligonucleotide
acaacucauuccagcccuagga

SEQ ID NO: 257 - RNA, 22 nt, Artificial
  Synthetic oligonucleotide
caacccucgagaauacggaaca

SEQ ID NO: 258 - RNA, 22 nt, Artificial
  Synthetic oligonucleotide
gacguccgcucauucucuaaua

SEQ ID NO: 259 - RNA, 22 nt, Artificial
  Synthetic oligonucleotide
acgccuacccgaaauguugccg

SEQ ID NO: 260 - RNA, 22 nt, Artificial
  Synthetic oligonucleotide
cacccacuauaauggcgggucg

SEQ ID NO: 261 - RNA, 22 nt, Artificial
  Synthetic oligonucleotide
gacggcgugccuaauugggucg

SEQ ID NO: 262 - RNA, 22 nt, Artificial
  Synthetic oligonucleotide
aaucuacaaugacuuagaaacg

SEQ ID NO: 263 - RNA, 22 nt, Artificial
  Synthetic oligonucleotide
gggaaccugagcccguaacgca

SEQ ID NO: 264 - RNA, 22 nt, Artificial
  Synthetic oligonucleotide
gucgguugauaacgccacaagc

SEQ ID NO: 265 - RNA, 22 nt, Artificial
  Synthetic oligonucleotide
cgaaaggacacuccuuccaaug

SEQ ID NO: 266 - RNA, 22 nt, Artificial
  Synthetic oligonucleotide
guuuaaagauauacggagcgau

SEQ ID NO: 267 - RNA, 22 nt, Artificial
  Synthetic oligonucleotide
cuuauuggguugcggugccaaa

SEQ ID NO: 268 - RNA, 22 nt, Artificial
  Synthetic oligonucleotide
gcgaguuagcuucgaaaccaau

SEQ ID NO: 269 - RNA, 22 nt, Artificial
  Synthetic oligonucleotide
ccguuaacaagucgcguaagag

SEQ ID NO: 270 - RNA, 22 nt, Artificial
  Synthetic oligonucleotide
cgccuaauauacguagaggcgg

SEQ ID NO: 271 - RNA, 22 nt, Artificial
  Synthetic oligonucleotide
guuacuucgauacgcccgacaa

SEQ ID NO: 272 - RNA, 22 nt, Artificial
  Synthetic oligonucleotide
ccuggccucagcuagccaauuu

SEQ ID NO: 273 - RNA, 22 nt, Artificial
  Synthetic oligonucleotide
aauauguggcuaucgccagccg

SEQ ID NO: 274 - RNA, 22 nt, Artificial
  Synthetic oligonucleotide
aaaugggcgggucgaauauaag

SEQ ID NO: 275 - RNA, 22 nt, Artificial
  Synthetic oligonucleotide
caucuauuaccaaccggaacau

SEQ ID NO: 276 - RNA, 22 nt, Artificial
  Synthetic oligonucleotide
gacacaaguggucuaucaccaa

SEQ ID NO: 277 - RNA, 22 nt, Artificial
  Synthetic oligonucleotide
gugucccgcgcuugagaacuag

SEQ ID NO: 278 - RNA, 22 nt, Artificial
  Synthetic oligonucleotide
aguagcccagagaaacauauca

SEQ ID NO: 279 - RNA, 22 nt, Artificial
  Synthetic oligonucleotide
aguaacuccauuucccggaccc

SEQ ID NO: 280 - RNA, 22 nt, Artificial
  Synthetic oligonucleotide
gcccacgagucguaaccauuaa

SEQ ID NO: 281 - RNA, 22 nt, Artificial
  Synthetic oligonucleotide
gcgccuuuggaguaaccacgcu

SEQ ID NO: 282 - RNA, 22 nt, Artificial
  Synthetic oligonucleotide
uccaacgaaacacgcuccgacg

SEQ ID NO: 283 - RNA, 22 nt, Artificial
  Synthetic oligonucleotide
acgccgacuuacgguuuaguaa

SEQ ID NO: 284 - RNA, 22 nt, Artificial
  Synthetic oligonucleotide
gacuuguucaaugaccgcccgg

SEQ ID NO: 285 - RNA, 22 nt, Artificial
  Synthetic oligonucleotide
cacuggucgggcccgaaacuug

SEQ ID NO: 286 - RNA, 22 nt, Artificial
  Synthetic oligonucleotide
ggauuuacauucgcccguguag

SEQ ID NO: 287 - RNA, 22 nt, Artificial
  Synthetic oligonucleotide
aacgcccaccugagucuuagcg

SEQ ID NO: 288 - RNA, 22 nt, Artificial
  Synthetic oligonucleotide
gccaacccgacguauucagaag

SEQ ID NO: 289 - RNA, 22 nt, Artificial
  Synthetic oligonucleotide
gccacgaggucccgaaaucgac

SEQ ID NO: 290 - RNA, 22 nt, Artificial
  Synthetic oligonucleotide
gcuacucguucccgccaacggg

SEQ ID NO: 291 - RNA, 22 nt, Artificial
  Synthetic oligonucleotide
ccgagcgucgaaguucguaaua

SEQ ID NO: 292 - RNA, 22 nt, Artificial
  Synthetic oligonucleotide
gaggccaaguuccuguaaccca

SEQ ID NO: 293 - RNA, 22 nt, Artificial
  Synthetic oligonucleotide
gguucgauaggagcguaacaaa

SEQ ID NO: 294 - RNA, 22 nt, Artificial
  Synthetic oligonucleotide
gggucgauauccaaagcccgca

SEQ ID NO: 295 - RNA, 22 nt, Artificial
  Synthetic oligonucleotide
ccacguucuaaaccgagacgcg

SEQ ID NO: 296 - RNA, 22 nt, Artificial
  Synthetic oligonucleotide
ggucuagucaccuuugaauucu

SEQ ID NO: 297 - RNA, 22 nt, Artificial
  Synthetic oligonucleotide
aucugacagguccgacucgcaa

SEQ ID NO: 298 - RNA, 22 nt, Artificial
  Synthetic oligonucleotide
guaaugggcgugguacccaucc

SEQ ID NO: 299 - RNA, 22 nt, Artificial
  Synthetic oligonucleotide
cuaucgugagcccgcuagacag

SEQ ID NO: 300 - RNA, 22 nt, Artificial
  Synthetic oligonucleotide
gucuggguuuaguucauaacgg

SEQ ID NO: 301 - RNA, 22 nt, Artificial
  Synthetic oligonucleotide
gcuguuaacuccuauagcaagu

SEQ ID NO: 302 - RNA, 22 nt, Artificial
  Synthetic oligonucleotide
aagcgauucguaacuauaaacg

SEQ ID NO: 303 - RNA, 22 nt, Artificial
  Synthetic oligonucleotide
ucgacucuacuuacccgaacga

SEQ ID NO: 304 - RNA, 22 nt, Artificial
  Synthetic oligonucleotide
ggguuuccuagccaaaucggaa

SEQ ID NO: 305 - RNA, 22 nt, Artificial
  Synthetic oligonucleotide
cgggccacacgcgcauuuaagc

SEQ ID NO: 306 - RNA, 22 nt, Artificial
  Synthetic oligonucleotide
ucucuuaagggaacggccaaag

SEQ ID NO: 307 - RNA, 22 nt, Artificial
  Synthetic oligonucleotide
gcaagccguaggcccacaaacg

SEQ ID NO: 308 - RNA, 22 nt, Artificial
  Synthetic oligonucleotide
cuggcgcucgcccuuacuuuau

SEQ ID NO: 309 - RNA, 22 nt, Artificial
  Synthetic oligonucleotide
cgcuacgaaauugguaaguaug

SEQ ID NO: 310 - RNA, 22 nt, Artificial
  Synthetic oligonucleotide
gggucagacauacgcaacggcu

SEQ ID NO: 311 - RNA, 22 nt, Artificial
  Synthetic oligonucleotide
gaagcgggccacaaaccuacuu

SEQ ID NO: 312 - RNA, 22 nt, Artificial
  Synthetic oligonucleotide
gagggcgauccuuacccguuua

SEQ ID NO: 313 - RNA, 22 nt, Artificial
  Synthetic oligonucleotide
ggucgauuuacauccgcguugu

SEQ ID NO: 314 - RNA, 22 nt, Artificial
  Synthetic oligonucleotide
cuaaccacuuacgggaguaaac

SEQ ID NO: 315 - RNA, 22 nt, Artificial
  Synthetic oligonucleotide
gagucccaacuucuaauuuagg

SEQ ID NO: 316 - RNA, 22 nt, Artificial
  Synthetic oligonucleotide
cccauauaccuuuauccgggua

SEQ ID NO: 317 - RNA, 22 nt, Artificial
  Synthetic oligonucleotide
ccgucacgacugaucaaccaga

SEQ ID NO: 318 - RNA, 22 nt, Artificial
  Synthetic oligonucleotide
ugauuaaggcccaauguucaaa

SEQ ID NO: 319 - RNA, 22 nt, Artificial
  Synthetic oligonucleotide
ccuaacgaugacggagguccac

SEQ ID NO: 320 - RNA, 22 nt, Artificial
  Synthetic oligonucleotide
ccuccagccgaugagucgguaa

SEQ ID NO: 321 - RNA, 22 nt, Artificial
  Synthetic oligonucleotide
gaaccggugcgcacgcuaagua

SEQ ID NO: 322 - RNA, 22 nt, Artificial
  Synthetic oligonucleotide
auugauccaaaccuuaacggac

SEQ ID NO: 323 - RNA, 22 nt, Artificial
  Synthetic oligonucleotide
gauggauaauccgggcuuaacu

SEQ ID NO: 324 - RNA, 22 nt, Artificial
  Synthetic oligonucleotide
gccuuuggacuaacccaaguac

SEQ ID NO: 325 - RNA, 22 nt, Artificial
  Synthetic oligonucleotide
ccgugguacccgcgccagcuag

SEQ ID NO: 326 - RNA, 22 nt, Artificial
  Synthetic oligonucleotide
cggucgcguaauuuccacacgu

SEQ ID NO: 327 - RNA, 22 nt, Artificial
  Synthetic oligonucleotide
cgccuuccgggaaagcccugcu

SEQ ID NO: 328 - RNA, 22 nt, Artificial
  Synthetic oligonucleotide
gcuuccguacguuauaucauuu

SEQ ID NO: 329 - RNA, 22 nt, Artificial
  Synthetic oligonucleotide
gccgcuuucgguuaaguccagg

SEQ ID NO: 330 - RNA, 22 nt, Artificial
  Synthetic oligonucleotide
cggucucaaacccgcguucuua